(19) United States
(12) Patent Application Publication
(10) Pub. No.: US 2022/0112351 A1
(43) Pub. Date: Apr. 14, 2022

(54) CHEMOLYTIC UPGRADING OF LOW-VALUE MACROMOLECULE FEEDSTOCKS TO HIGHER-VALUE FUELS AND CHEMICALS

(71) Applicant: Aduro Clean Technologies, Sarnia (CA)

(72) Inventors: W. Marcus Trygstad, Spring, TX (US); Anil K. Jhawar, London (CA)

(21) Appl. No.: 17/494,360

(22) Filed: Oct. 5, 2021

Related U.S. Application Data
(60) Provisional application No. 63/089,725, filed on Oct. 9, 2020.
(60) Provisional application No. 63/092,313, filed on Oct. 15, 2020.

(51) Int. Cl. C08J 11/10; C07C 269/08; B01J 19/24; C10G 1/10; C10G 1/04

(52) U.S. Cl. CPC C08J 11/10; C07C 269/08; B01J 19/245; C10G 1/10; C10G 1/04; C10G 2300/1011; C08J 2375/04; C08J 2367/02; C08J 2377/00; C08J 2371/00; C10G 2300/1003; B01J 2219/0004

(57) ABSTRACT

A method is provided for deconstructing macromolecules (MM) into lower molecular weight (MW) fragments in high yield by promoting first desirable reactions (Reactions1) that result in chemolytic scission of bonds in the backbone, chain, matrix, or network that defines the MM and obtain a first product mixture (Product1). The method includes conveying the prepared feedstock in a flowpath toward a reactor while adding a first agent of a first type (A1T1) suitable for promoting Reactions1, and a second agent (A2) suitable for promoting Reactions1 to obtain a first reaction mixture which is heated under controlled pressure.

### RELATED APPLICATION

This application claims the benefit of U.S. Provisional Patent Applications Ser. No. 63/089,725, entitled Chemolytic Upgrading of Low-Value Macromolecule Feedstocks to Higher-Value Fuels and Chemicals, filed on Oct. 9, 2020, and 63/092,313, entitled Chemolytic Upgrading of Low-Value Macromolecule Feedstocks to Higher-Value Fuels and Chemicals, the contents both of which are incorporated herein by reference in their entireties for all purposes.

### BACKGROUND

### Technical Field

This invention relates to hydrocarbon processing, and more particularly to systems and methods for efficiently producing high value products such as transportation fuels and chemical feedstocks.

### SUMMARY

Embodiments of the instant invention achieve upgrading of macromolecules (MM) by a system and methods that promote a plurality of desirable reactions including:

first desirable reactions that substantially deconstruct MM to obtain lower-molecular-weight fragments therefrom in high yield through the operation of first and second agents to obtain a first product mixture;

optionally, second desirable reactions that operate in the first product mixture to reduce levels of, or substantially eliminate, certain heteroatom-containing functionalities in fragments of certain MM, as applicable, and thereby obtain a second product mixture;

third desirable reactions that operate in first or second product mixtures, as the case may be, to quench reactive functionalities in MM fragments therein by means of hydrogen equivalents that render them substantially stable and unreactive, thereby obviating their participation in undesirable reactions that contravene outcomes from first or second desirable reactions and obtaining a third product mixture;

fourth desirable reactions that operate in first or second product mixtures, as the case may be, to effect in situ production, from third agents, of the hydrogen equivalents required in third desirable reactions; and

and optionally, fifth desirable reactions that operate on the third product mixture to substantially remove heteroatom-containing carboxylate functionality in components therein, which functionality persisted after the second desirable reactions.

The features and advantages described herein are not all-inclusive and various embodiments may include some, none, or all of the enumerated advantages. Additionally, many additional features and advantages will be apparent to one of ordinary skill in the art in view of the drawings, specification, and claims. Moreover, it should be noted that the language used in the specification has been principally selected for readability and instructional purposes, and not to limit the scope of the inventive subject matter.

### BRIEF DESCRIPTION OF THE DRAWINGS

The present invention is illustrated by way of example and not limitation in the figures of the accompanying drawings, in which like references indicate similar elements and in which:

FIG. 1 is a functional block diagram of aspects of embodiments of the present invention;

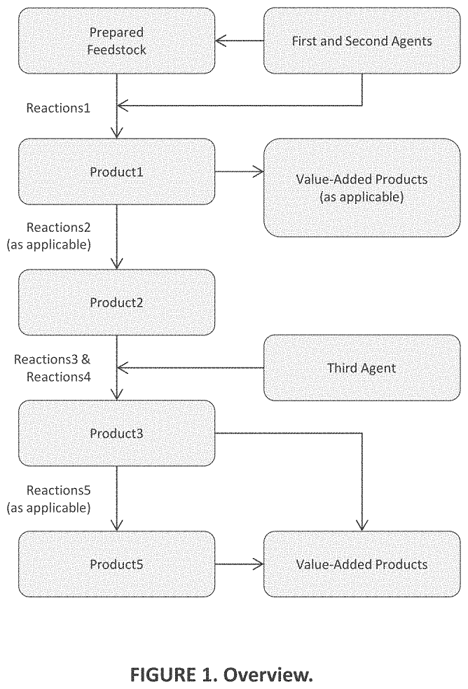

FIG. 2 is a functional block diagram of other aspects of embodiments of the present invention;

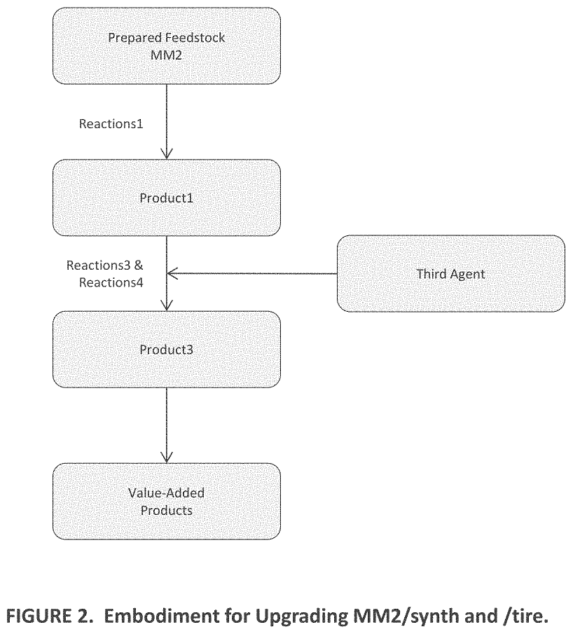

FIG. 3 is a view similar to those of FIGS. 1-2 of additional aspects of embodiments of the present invention;

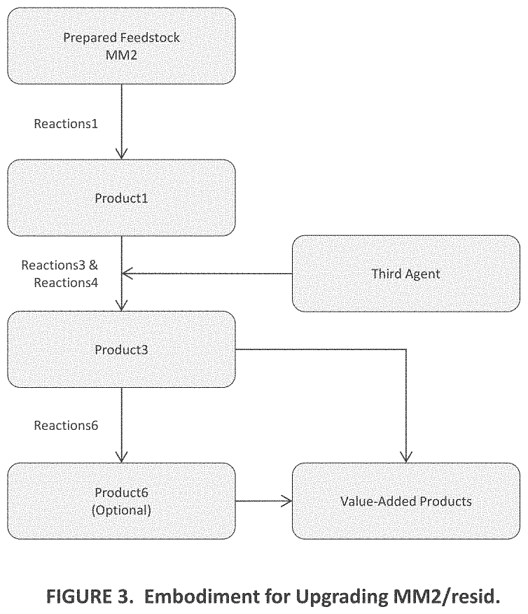

FIG. 4 is a view similar to those of FIGS. 1-3 of additional aspects of embodiments of the present invention;

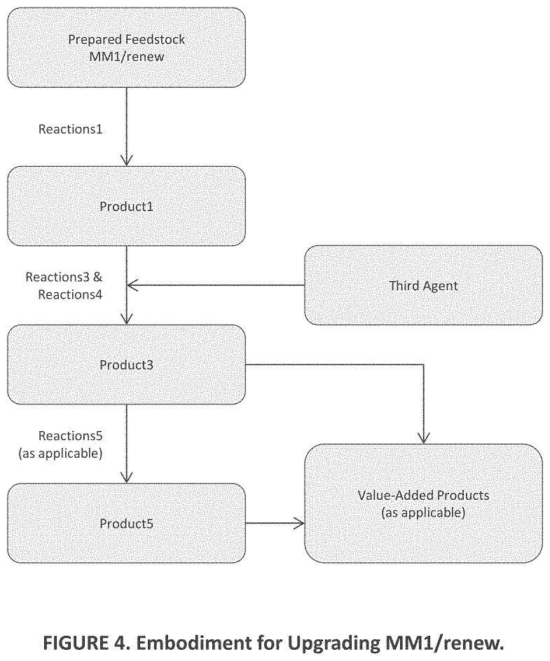

FIG. 5 is a view similar to those of FIGS. 1-4 of additional aspects of embodiments of the present invention;

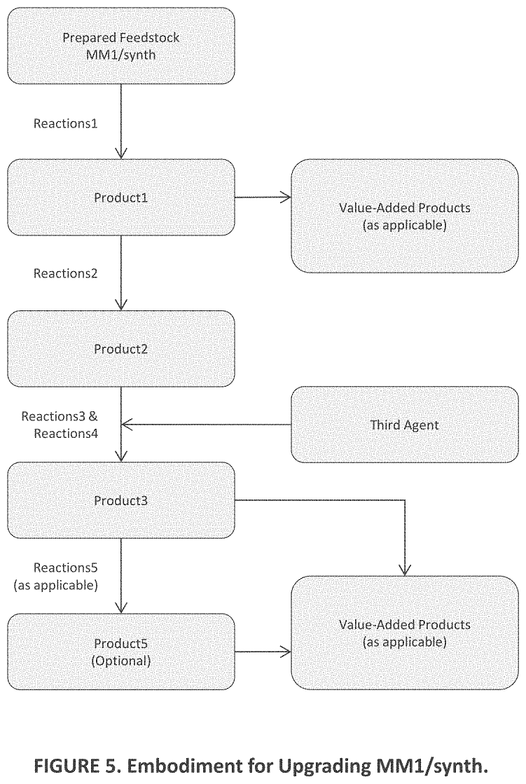

FIG. 6 is a view similar to those of FIGS. 1-5 of additional aspects of embodiments of the present invention;

FIG. 7 is a graphical representation of aspects of the embodiments of FIGS. 1-6; and

FIG. 8 is a diagrammatic representation of aspects of the embodiments of FIGS. 1-7.

### DETAILED DESCRIPTION

It should be understood at the outset that, although exemplary embodiments are illustrated in the figures and described below, the principles of the present disclosure may be implemented using any number of techniques, whether currently known or not. The present disclosure should in no way be limited to the exemplary implementations and techniques illustrated in the drawings and described below. Additionally, unless otherwise specifically noted, articles depicted in the drawings are not necessarily drawn to scale. In addition, well-known structures, circuits and techniques have not been shown in detail in order not to obscure the understanding of this description. The following detailed description is, therefore, not to be taken in a limiting sense, and the scope of the present invention is defined by the appended claims and their equivalents.

### General Overview

The instant invention relates to the upgrading of low-value macromolecules into higher-value products, where (i) macromolecules are high-MW molecules comprising substructures that may be homologous or heterologous, including by way of nonlimiting example, synthetic polymers contained in post-consumer plastics or foams and in rubber such as that in automobile tire; renewable materials such as cellulose, lignin, lignocellulose, and renewable oils; and heavy components in oil and bitumen such as asphaltenes; (ii) upgrading is by means of chemolysis designed to effect by means novel and nonobvious the limited deconstruction of the macromolecules into stable, lower molecular weight (MW) fragments whose chemical structures correspond nominally to those of the macromolecule substructures, where chemolysis occurs through the promoting of certain desirable chemical reactions while minimizing or preventing other reactions, as appropriate, that are undesirable; and (iii) higher-value products include by way of nonlimiting example hydrocarbons that may be used to produce transportation fuels or chemical feedstocks that may be used to coatings, lubricants, plasticizers, plastics, and rubber foams.

Macromolecules. In general, macromolecule is an imprecise term whose definition is not absolute, but is context-dependent and governed by considerations of chemical composition and structure, chemical properties, and physical properties. In particular embodiments, macromolecules may be regarded as organic compounds having more than about 20 carbon atoms covalently bonded within a single molecule or having boiling points (BP) corresponding to values greater than about 500° C. at standard temperature and pressure (STP). In other particular embodiments, the number of carbon atoms is greater than about 40 or BP may be greater than about 600° C. In yet other particular embodiments, feedstock macromolecules contain a number of carbon atoms in such great excess of 40 that their BP is indeterminate owing to the fact that even at reduced pressure they have substantially no vapor pressure and therefore undergo pyrolytic decomposition instead of distillation at elevated temperatures.

Also, the feedstock macromolecules may have structures that may be described variously as comprising a backbone, chain, matrix, or network, while in other cases the terms archipelago or continental are descriptive of the macromolecular structure. Certain thermoplastics comprising polymer molecules such as PE, PP, and PS comprise a saturated carbon backbone with varying but generally low amounts of chain branching or cross-linking. The term PE denotes its synthesis from ethylene monomers, but the final product may be more properly regarded as polymethylene insofar as it substantially comprises long chains of methylene groups. PP and PS differ from PE in that one out of every two carbon atoms bears a substituent that originates with the monomer from which the polymer was prepared, i.e. a methyl group in propylene and a phenyl group in styrene. PE, PP, and PS illustrate narrowly what is broadly true of all man-made polymers: they are formed through incorporation of lower-MW monomers into a single, high-MW macromolecule by way of reactions between the monomers that produce covalent bonds, the result being a solid material with a particular type of structure.

Macromolecules found in crude oil may seem initially to be unrelated to man-made polymers. For example, asphaltenes may be regarded as comprising an assemblage of molecular substructures that appear very diverse in composition and size and are covalently bonded to each other in seemingly innumerable ways. Yet, those substructures may in general be regarded as belonging to one of about six to eight broad categories whose members are closely related though not necessarily identical, and substructures of a given category comprise similar functional groups that are arranged similarly such that overall, their form, structure, or composition and the associated chemical properties are substantially similar. These building blocks are not monomers in the strict sense that the term connotes in polymer science, but the analogy is apt because the number of categories and their proportions are relatively limited, and within-category chemistries are similar. Consequently, in a given crude oil, the aggregate properties of asphaltene macromolecules assembled from them are likewise similar, just as are those of man-made polymers made from specific monomers, e.g., density, polarity, ratios of constituent elements, solubility, and chemical reactivity.

Asphaltenes may be regarded as a specific category of macromolecules at an extreme of the physico-chemical property continuum for crude oil components as regards molecular weight and polarity, where crude oil includes heavy crude oil and bitumen. In the common, coarse characterization of crude oils according to their saturate, aromatic, resin, and asphaltene content (SARA), asphaltenes differ from the rest in having relatively high polarity, which makes them substantially insoluble in nonpolar alkanes such as n-hexane. This also permits their production-scale separation from crude oils and bitumens by solvent deasphalting (SDA), a process that commonly employs an alkane solvent to dissolve low-polarity components, referred to generally as maltenes, while the asphaltenes precipitate. Yet, in a given crude oil, the demarcation between asphaltenes and higher-MW maltenes should not be regarded as abrupt. Rather, their chemical properties may be regarded as varying along a continuum. This point is established by the fact that the amount of asphaltenes rejected in SDA can vary significantly when the alkane varies within the series spanning butanes, pentanes, hexanes, and heptanes. The MW and polarity of asphaltenes is expected to be higher overall than for high-MW maltenes, and certain categories of substructures may be present at higher or lower levels in asphaltenes compared with those maltenes. But insofar as both may be regarded as crude oil macromolecules on which particular embodiments may operate, such materials will, for present purposes, be simply referred to as crude oil residues, residua, or resid(s) while the term asphaltene will be understood in the conventional sense as denoting those crude oil components that are insoluble in a defined alkane under conditions of defined time, temperature, and solvent-crude oil ratio.

Chemolytic Upgrading. In consideration of the constraints that limit the viability of thermolytic methods for upgrading macromolecules of interest, embodiments described herein derive significant benefit from chemolysis, which involves direct interplay between feedstock macromolecules and chemical agents in a reaction mixture. Wishing to contrast the two reaction types without being bound by particular theories of operation, thermolysis may be regarded principally as thermal-driven rupturing, or cracking, of covalent bonds within and between atoms defining the macro-structure of feedstock macromolecules to obtain smaller product molecules. Although employment of heterogeneous catalysis increases reaction rates, cracking may be regarded as a unimolecular process that cannot be describe as nuanced: reliance on high temperatures from 400° C. to as high as 1100° C. ensures the predominance of thermodynamic outcomes in which lowest-energy products are obtained at highest-possible rates with relatively limited possibilities for control of chemical outcomes.

Chemolytic upgrading is thought to differ in that bond scission occurs within feedstock macromolecules through direct interaction with, and by agency of, other chemical species included in a reaction mixture that is nominally single-phase. Although it also relies on the application of heat, the comparatively lower temperatures are thought to promote outcomes favored by kinetics rather than thermodynamics. Thus, embodiments operate below about 400° C., e.g., between about 225° C. and about 395° C., at which temperatures kinetic effects are thought to be substantially predominant. This means that products may form whose aggregate bond energies are higher than is possible for products obtained by thermolytic processes. For example, chemolysis is thought to deconstruct macromolecules into lower-MW fragments corresponding to their constituent substructures while leaving those substructures substantially intact. Thermolysis is similar to pyrolysis in that under their corresponding conditions, both operate substantially indiscriminately to break and rearrange bonds both between and within the substructures to obtain a mixture of products that have the lowest-possible aggregated bond energies and maximum entropy. Chemolytic processes in embodiments of the instant invention obtain a contrasting outcome wherein the aggregate bond energies of the product mixture, and the aggregate entropy of the same, are higher and lower, respectively, compared with products obtained by pyrolytic or thermolytic processes. As will be explained, benefits that accrue through chemolysis, compared with thermolytic and pyrolytic processes, relate to reduced energy requirements and the usefulness and yields of the products obtained.

Higher-Value Products. The macromolecule feedstock is the principal comparator in the assertion that embodiments yield higher-value products. The value differential becomes particularly dramatic in consideration of the fact that asphaltenes removed from Alberta bitumen by SDA, or plastics recovered from municipal solid waste streams (MSW), have low or negative value. That is, for the entity that possesses them, they represent a cost to be minimized. This is evident in the strategies discussed above whereby the low qualify of bitumen is offset by blending with diluent, or the compromised properties of recovered thermoplastics are overcome through co-melting with higher-quality virgin polymers. The value differential is further exacerbated in other scenarios discussed above, e.g., pyrolysis and gasification, whose product mixtures typically are in turn transformed by other processes into higher-value products. When the net capex and opex for such approaches is factored in, the possibility exists that the value uplift in products is very limited compared with the macromolecule feedstocks. Embodiments herein represent processes whose lower capital equipment and energy requirements obtain products with net higher value after upgrading feedstock macromolecules.

### Terminology

As used in the specification and in the appended claims, the singular forms “a”, “an”, and “the” include plural referents unless the context clearly indicates otherwise. For example, reference to “an analyzer” includes a plurality of such analyzers. In another example, reference to “an analysis” includes a plurality of such analyses.

Although specific terms are employed herein, they are used in a generic and descriptive sense only and not for purposes of limitation. All terms, including technical and scientific terms, as used herein, have the same meaning as commonly understood by one of ordinary skill in the art to which this invention belongs unless a term has been otherwise defined. It will be further understood that terms, such as those defined in commonly used dictionaries, should be interpreted as having a meaning as commonly understood by a person having ordinary skill in the art to which this invention belongs. It will be further understood that terms, such as those defined in commonly used dictionaries, should be interpreted as having a meaning that is consistent with their meaning in the context of the relevant art and the present disclosure. Such commonly used terms will not be interpreted in an idealized or overly formal sense unless the disclosure herein expressly so defines otherwise.

Referring now to the Figures, embodiments of the present invention will be described in detail.

Initially, aspects of the invention include the realization by the instant inventors that each day vast quantities of durable products made from plastic, foam, synthetic fibers, and rubber are discarded at end-of-life; the same is true for single-use packaging made of plastic and foam. Much of it flows into managed municipal solid waste (MSW) streams from which it may be recovered for recycling while the rest many be landfilled, incinerated, or dumped into oceans by the unscrupulous. Similar fates await used tires, but they represent a distinct subset of the problem that generally may be managed separately. Clearly, the quantities and ubiquity owe principally to the high benefit-cost ratio of articles made of synthetic polymers, where both cost and benefit regard only production and use but excludes post-use considerations. While the problems they cause owe to multiple and diverse factors, the high benefit and low front-end cost arguably serve to effectuate those factors. For example, the benefit relates principally to the versatility, which derives from mechanical and chemical properties that can be engineered to make polymeric materials formable and durable. Durability ensures that articles made from man-made polymers are fit for purpose, from seat cushions and mattresses to containers for food and beverages; its availability at relatively low front-end production costs ensures high demand and, hence, large quantities. Yet, the very durability that makes man-made polymeric materials so useful also makes them highly problematic in nature: because they resist rapid decomposition by chemical or biological means, they accumulate on land, or in the oceans where miniscule polymer particles formed by mechanical processes infuse the very tissues of sea creatures living there.

As-Is Reuse. Thinking and practice regarding the recovery and reuse of polymeric materials has been informed by and predicated on this durability. The guiding concept is basically the same as that which underlies recycling of other durable materials such as steel, aluminum, glass, and paper: gather; segregate, and purify as appropriate; then blend with virgin material for resuse in production of new articles. For common thermoplastic polymers like polyethylene (PE), polypropylene (PP) and poly(ethyleneterephthalate) (PETS), this generally involves melting and blending with virgin polymer to mitigate to the variable quality and/or degradation of properties incurred through use and recovery. Tire rubber and polyurethane foam differ in that they are thermoset polymers that cannot be physically melted and re-formed into new articles; recycling is instead limited generally to blending of polymer granules with other materials to make useful products, e.g., paving asphalt in the case of tire rubber and new rubber foam in the case of polyurethane. In all instances, reuse of plastics and rubber is governed by the Goldilocks principle: percent recycled polymer in the blend must be high enough for the recycling strategy to be relevant but low enough to avoid unacceptable compromise of properties in the finished product. However, this implies that in the limit, recycling strategies suitable for metals, glass, and paper represent at best a partial solution because at some high rate of post-use recovery, the amounts of recovered plastic and rubber will exceed what can be blended for reuse.

Thermo-Chemical Methods. At the other extreme, a decades-old approach called gasification has gained renewed attention. Operating at high temperatures, the process involves the controlled addition of a limited amount of oxygen (from air) to convert the elements in organic feedstocks into syngas (synthetic gas) comprising molecular hydrogen, carbon monoxide, and some carbon dioxide. For example, gasification has been applied to diverse material ranging from coal and biomass to municipal solid waste and post-consumer plastic. Syngas in turn can be used to produce hydrogen, ammonia, methanol, and synthetic hydrocarbon fuels by long-established methods.

Another approach garnering attention despite also being antiquated is pyrolysis, which also applies high temperatures but generally excludes oxygen. Conditions applied result in non-oxidative thermolysis whereby bonds are broken and rearranged, and molecular fragments are transformed into a mixture of diverse products that may bear limited resemblance to the starting material and subsequently may be post-processed, e.g., subjected to variety of purification steps, to ensure suitability as platform chemicals, feedstocks in chemical manufacturing, and fuels. Though the gasification and pyrolytic approaches differ significantly, each may be regarded as the opposite of the reuse-by-blending approach. The latter has relatively low energy requirements, leaves the macromolecules intact, and seeks to reuse the polymers for purposes similar to those for which they were originally made, tire rubber being an exception. By contrast, the other approaches are energy-intensive, and they decompose and rearrange substructure functionalites of feedstock polymers into smaller molecules whose chemical nature and uses are largely unrelated to those of the starting material. Yet, the three approaches are similar in that their broad relevance is constrained by factors grounded in economics and/or chemistry.

Petroleum Refining. Petroleum refining suggests a possible alternative that lies between these extremes. Refineries are complex and highly integrated assemblages of diverse operating units that employ long-established technologies to efficiently transform crude oil into transportation fuels, lubricants, and feedstocks to other petrochemical plants. Particularly relevant to the present discussion are operating units that employ thermal cracking, often in combination with added hydrogen, to break apart heavy macromolecules contained in the petroleum feed, obtaining thereby lighter hydrocarbon molecules suitable for gasoline and diesel, where the terms “light” and “heavy” are synonyms for low and high molecular weight (MW), respectively. Catalytic cracking and hydrocracking are notable examples, their implementation in the context of refining being practical and economically favorable due to the availability of infrastructure that supports the efficient processing of whole crude oil, not just the problematic heavy fraction.

Bitumen Upgrading. This contrasts the situation at the other end of the oil pipeline, for example, in Alberta, Canada. There, instead of refining into fungible fuels, the more limited objective is to upgrade the properties of bitumen produced from abundant oil sands deposits, the principal concern being to decrease density and viscosity to levels that permit transportation through oil pipeline, and the principal obstacle being the heavy asphaltenes, whose levels may be as high as 25%. An approach favored on grounds of low cost and complexity is directly analogous to the recycling of thermoplastics; it involves blending bitumen with lighter, higher-value hydrocarbons in bitumen-hydrocarbon ratios between about 8:1 and 2:1. In this production of diluted bitumen (dilbit), the Goldilocks ratio is that which maximizes the blend ratio to minimize usage of expensive diluent while also meeting the pipeline specifications, 3:1 being typical. Though regarded as upgrading due to the focus on bitumen, this overlooks the concomitant, ironic downgrading of the diluent. Additional disadvantages attached to upgrading-by-dilution include: reduction in the amount of actual bitumen carried by a given pipeline, e.g., by 25% when the blend ratio is 3:1; the cost and availability of diluent.

The upstream problem may alternatively be solved through true upgrading based on application of more sophisticated downstream methods employed in oil refining to transform the chemical composition of heavy components in bitumen, especially asphaltenes. The qualities of synthetic crude produced this way typically exceeds that of dilbit, having lower values for properties such as sulfur, microcarbon residue, acid values, and metals content. But instead of being traded outright, the synthetic crude is commonly combined with diverse crudes, bitumens, and diluent to produce a grade of crude oil with defined, consistent properties, e.g., Western Canadian Select (WCS).

Bitumen Upgrading and Plastic Upcycling: Similar Issues. Upon initial consideration, achieving the conversion of plastic and rubber into higher-value materials might seem to represent a problem unrelated to upgrading of asphaltenes. But they have two issues in common: the objective of creating materials of higher value from lower-value starting materials; and the limited viability of doing so by leveraging refining or thermolytic methodologies, or upgrading-by-dilution. Still, a mature refining technology such as hydrocracking suggests a desirable feature of a middle path for upgrading plastics, rubber, or asphaltenes, one that lies between the aforementioned extremes of recycling by dilution for as-is use and gasification or pyrolysis. In particular, it points to their partial breakdown into lower-MW product molecules directly related chemically to the feedstock. Rather than total decomposition of the hydrocarbon framework accomplished by gasification and pyrolysis, the products are fragments that retain some chemical characteristics of the parent macromolecules. For some man-made polymer feedstocks, they may be fragments of a polymer chain; for others, they may be the very monomers from which the polymer was synthesized, or perhaps derivatives of those monomers. And the situation is analogous for macromolecules in bitumen such as asphaltene, which may be regarded as comprising submolecular structures linked to each other. Thus, the product molecules would in concept derive from those substructures.

Possibilities and Limitations of Hydrocracking. By way of intention to illustrate but not be limited by a particular theory of hydrocracker operation, it may be regarded generally as promoting two chemical processes facilitated by a heterogeneous catalyst. The first begins with thermally-driven homolytic scission (thermolysis) of a covalent bond between two carbon atoms within or between macromolecule substructures, whereby the bonding electron pair is divided between the two atoms to yield two reactive free radicals. Those may persist as transient intermediates or undergo relatively rapid, reactions involving inter-molecular hydrogen transfers to yield, by way nonlimiting example, methyl or olefinic end-groups. The unsaturated functionality is reduced by addition of atomic hydrogen, supplied to the process as molecular hydrogen (H₂) generated elsewhere within the refinery. Those skilled in the art will appreciate (i) the possibility for other modes of thermolytic bond scission, e.g., heterolysis; and (ii) the presence within an asphaltene molecule of other atoms such as nitrogen and sulfur whose bonds are likewise susceptible to being broken and/or reduced by a variety of mechanisms. The example of homolysis serves nonetheless to illustrate paired features of hydrocracking: the thermolytic decomposition of feedstock macromolecules into lower-MW fragments; and, when those fragments are reactive intermediates, their reduction with hydrogen to form stable products.

Highly valuable in petroleum refining, hydrocracking is representative of established processes that in concept could be adapted to great benefit as an alternative for upgrading end-of-life plastics and rubber. However, the obstacles are formidable. One is development of a heterogeneous catalyst that is suitable for promoting the desired chemistry while resisting deactivation by feed impurities. But perhaps the most important relates to efficiencies achieved within petroleum refineries through an ensemble of diverse conversion units that operate as a highly integrated whole while offering a modicum of operational flexibility within limits that permit conversion of somewhat variable feedstocks into diverse products ranging from hydrocarbon fuels and lubricants to petrochemical feedstocks and road asphalt. The same favorable economics apparently do not accrue when related technologies are applied to obtain a single product from a single feed, i.e. heavy crude oil from heavier bitumen. That reality may likewise limit the commercial viability of repurposing refining technology for upgrading of post-consumer plastic, foam, and rubber. Significantly, chemical technology for decomposing poly(ethyleneterephthalate) (PET) and polyurethane (PU) foams into the chemical building blocks from which they were synthesized was available well before 2000. Yet this not widely practiced, suggesting economics as the limiting issue, perhaps because the products lack chemical purity or homogeneity, and/or they lack value sufficient to offset the associated costs.

More specifically, the need exists for an efficient, economical, and flexible system and method to convert lower-value macromolecules such as plastics, rubber, components in heavy oil and bitumen, renewable oils, and biomass-based materials into higher-value products comprising stable, lower-MW fragments of the feedstock macromolecules. Recognizing this need, the inventors of the instant invention conceived, developed, and now describe an altogether novel and nonobvious system and method that represents a contemporary solution to these contemporary challenges.

### Embodiments

Embodiments of the present invention provide a system and method comprising the following: Promoting first desirable reactions that deconstruct MM to obtain a first product mixture containing lower-molecular-weight fragments thereof in high yield by,
(a) Configuring a flowpath to receive a prepared feedstock in the form of a powder, a liquid, granules, a suspension, a slurry, or a solution containing one or more materials from the group consisting of, but not limited to, MM of a first kind, which include certain plastics and foams, lignin, lignocellulosic materials, renewable oils, and biomass, and MM of a second kind, which include certain other plastics and foams as well as rubber, heavy oil, and resid;
(b) Conveying the prepared feedstock in a flowpath toward a reactor;
(c) Contacting the prepared feedstock in the flowpath before the reactor or in the reactor with a first agent of a first type (A1T1) to obtain a reaction premixture;
(d) Further contacting the premixture in the flowpath before the reactor or in the reactor with a second agent (A2) to obtain a first reaction mixture;
(e) Optionally configuring the flowpath to preheat the prepared feedstock and/or the reaction premixture and/or first reaction mixture flowing therethrough, as the case may be, to a temperature of up to a first temperature T1/max before being conveyed into the reactor;
(f) Configuring the reactor to receive and heat the first reaction mixture to a temperature range T(range)1 in the range between T1/min and T1/max for a length of time t1;
(g) Selecting A1T1 and T(range)1 in respect of MM chemistry and kind, which determine MM susceptibility to undergo first desirable reactions; and
(h) Controlling the relative amounts of A1T that exist in the liquid and gas phases in the reactor at levels suitable to promote first desirable reactions, when the first reaction mixture is heated in the reactor to T(range)1, by configuring the reactor to control pressure in the same, and by controlling the relative volumes of liquid and headspace in the reactor, and by controlling the relative amounts of A1T and MM contacted in the flowpath before the reactor or in the reactor.

Additional optional aspects include contacting the prepared feedstock in the flowpath, before the reactor and before contacting with A1T1, with a first agent of a second type (A1T2) to obtain a premixture, where A1T2 and its quantity are selected to predispose the prepared feedstock to contacting by A1T1 and A2, and also to undergoing Reactions1;

isolating from the first product mixture certain higher-value components when the prepared feedstock contains certain MM of a first kind, where the higher-value components are chemicals suitable for use in production of added value products;

recovering A1T2 from the first product mixture for reuse in production of the premixture; and

promoting second desirable reactions in the first product mixture, which substantially eliminate certain heteroatom-containing functional groups from fragments of certain MM of a first kind, by further heating the first product mixture in the reactor to a temperature range T(range)2 in the range between T2/min and T2/max for a time t2 to obtain a second product mixture, where T2/min≥T1/min and T2/max≥T1/max;

Promoting third desirable reactions that substantially quench reactive functionalities of in MM fragments in first and/or second product mixture, as the case may be, to obtain a third product mixture, where the quenching occurs by agency hydrogen equivalents generated in situ from a third agent that undergoes a fourth desirable reaction, the third and fourth desirable reactions are promoted by further heating the reaction mixture in the reactor to a temperature range T(range)3 in the range between T3/min and T3/max in the presence of a third agent for a time t3, and where T3/min≥T2/min and T3/max≥T2/max, and where the third agent is in the reaction mixture by dint of its formation via first desirable reactions involving certain first MM of a first kind in the prepared feedstock and/or where the third agent is added to the reaction mixture;

promoting fifth desirable reactions, which substantially eliminate carboxyl functionality from fragments of certain MM of a first or second kind, by further heating the third product mixture in the reactor to a temperature range T(range)5 in the range between T5/min and T5/max for a time t5 to obtain a fifth product mixture, where T5/min≥T3/min and T5/max≥T3/max, and where the eliminating of carboxyl functionality is decarboxylation or decarbonylation; and

Isolating higher-value products from the third or fifth product mixtures, as the case may be, by standard methods including one or more taken from the group including but not limited to separation of liquid phases, precipitation, and distillation;

wherein the promoting of the second, third, fourth, and fifth desirable reactions in the corresponding product mixtures, as applicable, is accomplished in a reactor configured to progressively heat reaction mixtures flowing therethrough, e.g., from T(range)1 to T(range)2 to T(range)3 to T(range)5, and where the reactor may in certain embodiments comprise an ensemble of reactor sub-sections communicably-coupled in series and differentiated according to temperature; or optionally the desirable reactions take place in a single reactor operating at a T(range) suitable to progressively promote all the aforementioned desirable reactions.

Embodiments that Promote Reactions1. Particular embodiments are a system and method that promote first desirable reactions (Reactions1) in a prepared feedstock containing macromolecules (MM), which reactions are chemolytic reactions that deconstruct MM to obtain lower-MW fragments therefrom in high yield, by (i) conveying the prepared feedstock in a flowpath toward a reactor; and (ii) contacting the prepared feedstock in the flowpath before the reactor or in the reactor with a first agent of a first type (A1T1), which promotes Reactions1; and (iii) further contacting the prepared feedstock in the flowpath before the reactor or in the reactor with a second agent (A2) to obtain a first reaction mixture (Mix1) comprising the prepared feedstock, A1T1 and A2; and (iv) optionally contacting the prepared feedstock in the flowpath, before contacting it with A1T1 and A2, with a first agent of a second type (A1T2) to obtain a premixture; and (v) configuring the reactor to receive and heat the prepared feedstock or the premixture from the flowpath, as the case may be; and (vi) optionally configuring the flowpath to preheat the prepared feedstock or the premixture or Mix1 flowing therethrough, as the case may be, to a temperature of up to T1/min before being conveyed into the reactor; and (vii) heating Mix1 in the reactor to a temperature range T(range)1 in the range between about T1/min and about T1/max for a length of time t1; and (viii) selecting A1T1, T(range)1, and t1 in respect of MM susceptibility to undergo Reactions1 which is a function of MM chemical composition; and (ix) selecting the total amount of A1T1 in Mix1, and configuring the reactor to control the total pressure therein, to establish amounts of A1T1 that exist in the liquid and gas phases when the first reaction mixture is heated in the reactor to T(range)1 where the amounts of the first agent in the two phases are selected in respect of MM chemistry and kind and are sufficient to support Reactions1.

High yield refers to an extent of MM deconstruction that obtains products that may be regarded as consisting substantially of condensed-phase compounds corresponding to constituent substructures of MM from which they are derived while minimizing or avoiding decomposition into low-MW compounds that may be regarded as byproduct gases, where the latter include diatomic compounds such as H₂ and CO, triatomic compounds such as CO₂, and hydrocarbons containing one to about four carbon atoms, e.g., methane, ethane, ethylene, propane, propylene, butanes, and butylenes; and where the extent of MM deconstruction is controlled in particular embodiments through their configuration with respect to temperature, time, and the first and second agents.

In the particular embodiments that promote Reactions1, the prepared feedstock contains one or more materials from the group consisting of, but not limited to, MM of a first kind (MM1) that include but are not limited to certain plastics and foams, lignin, cellulosic and lignocellulosic materials, renewable oils, and biomass, and MM of a second kind (MM2) that include certain other plastics and foams, as well as rubber, heavy oil, and resid; the prepared feedstock consists of powders, granules, suspensions, slurries, solutions, or liquids that contain the MM; the optional first agent of a second type (A1T2) is a hydrocarbon; the MM-A1T2 premixture is a suspension, a slurry, or a solution; the first agent of a first type (A1T1) is a protic solvent or compound; the net MM-A1T2 ratio in Mix1 is between about 1:4 and 4:1 and the MM-A1T1 ratio in Mix1 is between about 10:1 and 1:10; A2 contains one or more metals in compounds with the general formula (Mᵢ)ₐX_{b}; the concentration [Mᵢ] of a metal Mᵢ in Mix1 is between about 10 and about 250 milliequivalents (meq) per kg MM and the total concentration of metals Σ[Mᵢ] is between about 30 and about 750 meq per kg MM, where an equivalent is a molar equivalent; T(range)1 has minimum and maximum values of T1/min and T1/max, respectively, and is in the range between about 225° C. and about 375° C.; and t1 is between about 10 minutes and 250 minutes. Nonlimiting examples of A1T2 include one or more hydrocarbon compounds taken from the group consisting of alkanes, cycloalkanes, and aromatics, where alkanes and cycloalkanes have the general formulas CₙH₂ₙ₊₂ and CₙH₂ₙ, respectively, and n is between about 5 and 20; cycloalkanes include substituted cycloalkane moieties bearing zero or more alkyl substituents; and aromatics are mono-, di-, or trisubstituted benzene compounds where the substituents are alkyl groups containing from one to about four carbon atoms. Nonlimiting examples of A1T1 include water; alcohols containing up to about four carbons, including diols; and alkyl amines containing up to about six carbons. In A2 compounds (Mᵢ)ₐX_{b}, the metals Mᵢ include one or more taken from groups 3-14 of the periodic table of chemical elements, nonlimiting examples of which include yttrium from group 3, titanium from group 4, vanadium from group 5, molybdenum from group 6, manganese from group 7, iron from group 8, cobalt from group 9, nickel from group 10, copper from group 11, zinc from group 12, aluminum from group 13, and tin from group 14; the oxidation state of the metal is +m; and X is any simple or complex anion whose charge has an integer value x=−[a/b·(+m)] and may include by way of nonlimiting example one or more taken from the group consisting of oxide (O⁻²), sulfate, bisulfate, nitrate, chloride, carbonate, bicarbonate, acetate, and any alkyl carbanion.

First Desirable Reactions. Though wishing not to be constrained by any particular theory of operation, Reactions1 occurring in particular embodiments are thought to promote the aforementioned deconstruction through one or more chemical effects including but not limited to: (i) the increasing of ion product (the extent of proton dissociation) for A1T1 at elevated temperatures, e.g., above about 290° C.; (ii) the operation of dissociated protons and/or the corresponding counterions from A1T1 to catalyze Reactions1; (iii) the decreasing of dielectric constant, viscosity, and surface tension of A1T1 as T increases; (iv) the increasing of the diffusivity of first agents A1 as a function of T; (v) the promoting of the disruption and disaggregation MM aggregates and/or matrices through infusion of first agents A1 into the same through effects (iii) and (iv); (vi) the predisposing of MM to undergo Reactions1 by the operation of effect (v); (vii) the enhancing of the aforementioned effects through the operation of A2; and (viii) the maximizing of the aggregate operation of effects (i)-(vii), and the maximizing thereby of Reactions1 rates, through selection of temperatures in respect of the MM kind and chemical composition. The first, second, and third effects enumerated are not singularly important, but highlight the beneficial changes in properties of A1T as temperature increases. For example, the pK_{w} of water decreases from 14 at 25° C. to 11.2 at about 250° C.-300° C., meaning that hydrogen ion and hydroxide ion each is 300 times higher at the higher temperature; and the dielectric constant of water decreases from 80 to 6 across a similar temperature range. In summary, particular embodiments promote increased susceptibility of MM to undergo Reactions1 through the synergistic operation of first and second agents at T(range)1 selected in consideration of MM chemical composition.

Feedstock: MM1. The feedstock in particular embodiments includes MM1 that substantially consist of monomeric or monomer-like substructures, or chemically similar substructures, linked to each other through functionality in which bonds between one or more heteroatoms define the MM backbone, chain, matrix, or network, e.g., nitrogen and/or oxygen. When MM1 is a synthetic polymer (MM1/synth), nonlimiting examples include materials comprising a backbone, chain, matrix, or network in which the linkages are esters, urethanes, or amides formed through step-growth polymerization, or ethers formed through chain reaction (addition polymerization) mechanisms. When MM1 consists of renewable materials, nonlimiting examples of linkages between substructures include ester, ether, acetal, hemiacetal, hemiketal, peptide functionalities; and when MM1 is renewable oils, the linkages are esters formed between fatty acids and glycerol. In the chemolytic deconstruction of MM1 by Reactions1, A1T1 molecules add across the heteroatom linkages such that they are incorporated into the lower-MW macromolecule fragments. Generally referred to as solvolysis, solvolytic depolymerization, or solvothermolysis, the specific terms hydrolysis and hydrochemolysis apply when A1T is water, and the terms alcoholysis and aminolysis apply when A1T1 is an alcohol or an amine, respectively.

For purposes of the instant invention, step-growth polymerization by which certain MM1/synth are formed shall be understood to include step-growth polyaddition polymerization, the former term connoting that reactants are simple monomers while in the latter, one or more of the components used to synthesize MM1 is a prepolymer, e.g., a material which has a molecular weight intermediate between that of simple monomers and the polymer product and is itself produced from one or more monomeric materials. In particular embodiments, MM1/synth formed through step-growth polymerization include materials comprising, containing, or made from one or more taken from the group including but not limited to, (i) polyesters obtained by condensation polymerization of polyhydric alcohols, e.g., diols or polyols, with dicarboxylic acids or esters thereof; or (ii) polyurethanes (PU) obtained by step polymerization of polyhydric alcohols with diisocyantes; or (iii) polyamides obtained by condensation polymerization of polyamines with dicarboxylic acids or esters thereof, or by addition polymerization of aminocarboxylic acids or their corresponding lactams.

Nonlimiting examples of polyesters include: poly(ethylene terephthalate) (PET), synthesized from ethane-1,2-diol (ethylene glycol, or EG) and terephthalic acid or dimethyl terephthalate (DMT); other poly(alkylene terephthalate) compounds in which EG is replaced by diols with the general formula, (CₓH_{y})(OH)₂, where y=2x or 2x−2 and alkylene includes by way of nonlimiting example trimethylene, butylene, and cyclohexenedimethylene; and polyester resins formulated from one or more polyhydric alcohols and from polybasic carboxylic acids containing at least two carboxyl groups, or from esters thereof, e.g. methyl esters or ethyl esters. Nonlimiting examples of PU include those formulated from one or more diverse polyhydric alcohols and/or pre-polymeric polyols and from diisocyantes, examples of the latter including but not limited to methylene diphenyl diisocyanate (MDI) and toluene diisocyanate (TDI).

Nonlimiting examples of polyhydric alcohols used to produce polyesters include one or more taken from the group including but not limited to those with the formulas: CₓH_{(2x+2-y)}(OH)_{y}, which includes diols, triols, and tetrols (y=2, 3, and 4, respectively, and x≥y) as well as sorbitol (x=y=6) and sorbitan (x=6, y=5); HO(CₓH₂ₓO)ₙH, which includes polyoxyalkylene diols; and hydroxyl-terminated polyoxyalkylene adducts of any of diols, triols, or tetrols, or of sorbitan or sorbitol. Nonlimiting examples of polyoxyalkylenes include: polyoxymethylene, polyoxyethylene, polyoxypropylylene, and polyoxybutylene (x=1, 2, 3, and 4, respectively, and n has values from about 1 to about 15); mixed polyoxyalkylenes made by co-polymerization of two or more alkylene oxides, e.g. of ethylene oxide and propylene oxide such that 2<x<3; and low-MW polyoxyethylene homologs such as diethylene glycol and triethylene glycol (x=2, and n=2 and 3, respectively) and their propylene-based counterparts (x=3). Polyhydric alcohols used to produce PU include one or more taken from the group including but not limited to those enumerated above in connection with polyester resins; and polyester resins described hereinabove, formulated with a number of hydroxyl equivalents from polyhydric alcohols that exceeds the number of acid equivalents polybasic carboxylic acids such that condensation polymerization yields hydroxyl-terminated polyols. Nonlimiting examples of polybasic carboxylic acids include compounds or mixtures of compounds the general formula (CₓH_{y})(COOH)_{z}, where x typically has values of about 1 to about 40, y=2x for saturated compounds, y≤2x for unsaturated compounds, and z typically has values of 2 to about 3.

Common dicarboxylic acids include those with the formula HOOC(CH₂)ₙCOOH, where n typically has values from about 1 to 16; and also dimer fatty acids produced by catalyzed dimerization of fatty acids containing between about 14 and about 22 carbons, e.g., by dimerization oleic acid, (C₁₇H₃₃)COOH, to form a dicarboxylic acid whose formula is nominally HOOC(C₃₄H₆₆)COOH.

Nonlimiting examples of polyamides include diverse materials referred to commonly as nylon, which include: nylons-c,d such as nylon-4,6, nylon-6,6, nylon-6,9, nylon-6,10, nylon-6,12, and nylon-10,10, which are prepared by condensation polymerization between diamines H₂N(CH₂)_{c}NH₂ (4≤c≤10) and diacids HOOC(CH₂)_{d-2}COOH (6≤d≤12) to obtain polymers with the general formula, [NH(CH₂)_{c}NHCO(CH₂)_{d-2}CO]ₙ; and also nylons-e such as nylon-3, nylon-6, nylon-8, nylon-10, nylon-11, prepared by condensation polymerization of aminocarboxylic acids with the general formula HOOC(CH₂)ₑ₋₁NH₂ (3≤e≤12), or by addition polymerization of their corresponding lactams, to obtain polymers with the general formula, [NH(CH₂)ₑ₋₁CO]ₙ.

Chemolytic Deconstruction of MM1/synth. In particular embodiments that promote Reactions1 to deconstruct MM1/synth, A1T1 consists of one or more materials from the group including but not limited to: water; an alcohol such as methanol, ethanol, ethane-1,2-diol, propane-1,2,3-triol, butane-1,4-diol, and the like; and amines including mono- and di-alkyl amines such as methylamine, ethylamine, dimethylamine, diethylamine, and the like. Compared with prior art for depolymerization of MM1/synth such as PET and PU, particular embodiments that effect chemolysis in MM1/synth are unique and nonobvious in respect of A2, which may operate by advantageously enhancing rates of Reactions1 to achieve desired outcomes more quickly and/or at lower temperatures while avoiding certain undesirable reactions that otherwise may occur at elevated temperatures. But as will now be explained, the importance of such embodiments resides in subsequent transformations of chemolysis products by additional desirable reactions that support production of higher-value fuels and chemicals.

Particular embodiments promote deconstruction of MM1/synth to obtain a first product mixture (Product1) by operation of Reactions1 on heteroatom linkages which, in the case of MM1/synth, originally formed through the step-growth polymerization that obtained the polymer backbone, chain, matrix, or network. Those embodiments yield MM fragments corresponding to components that were combined to form MM1/synth in the feedstock, as depicted in Table I, including but not limited to: (i) polyhydric alcohols and dicarboxylic acids or esters of the latter, which were combined through condensation polymerization to obtain polyesters; or (ii) polyhydric alcohols and diisocyantes, which were combined through addition polymerization to form PU; or (iii) diamines and dicarboxylic acids or esters of the latter, or aminocarboxylic acids or their corresponding lactams, which were combined to form polyamides through condensation and addition polymerization reactions, respectively. Thus, except for diisocyanates used in production of PU, chemolytic deconstruction of MM1/synth yields in Product1 substantially the very components that were combined to make them. Isocyanates yield instead the corresponding amines according to reactions (1) and (2), where the net result is equivalent to the well-known reaction of isocyanate with water according to reaction (3).

### Reactions Relating to Formation and Chemolytic Deconstruction of Polyurethanes.

### Urethane Formation

ROH+R′NCO→RO(CO)NHR′ (1)

### Hydrochemolysis of Urethane

RO(CO)NHR′+H₂O→ROH+R′NH₂+CO₂ (2)

Isocyanate Reaction with Water

R′NCO+H₂O→R′NH₂+CO₂ (3)

TABLE I
Examples of Components in MM1/synth and Products1 from Reactions1.

| First | | | |
|---|---|---|---|
| Macro- | Polyesters and | | |
| molecule | Polyester Resins | Polyurethanes | Polyamides |
| Components | Polyhydric Alcohols, | Polyhydric Alcohols, CₓH_{(2x+2-y)}(OH)_{y} | Dibasic Carboxylic Acids, |
| Used to | CₓH_{(2x+2-y)}(OH)_{y} Polybasic | Diisocyanates, e.g., TDI (CH₂(C₆H₄NCO)₂) | (CₓH₂ₓ)(COOH)₂, |
| Synthesize | Carboxylic Acids, (CᵤH₍₂ᵤ₊₂₋ᵥ₎)(COOH)ᵥ | and/or MDI (CH₃(C₆H₃)(NCO)₂) | and Diamines, e.g., (CᵤH₂ᵤ)(NH₂)₂; or |
| the Macro- | where y > 1 and v > 1 | | Aminocarboxylic acids, |
| molecule | | | HOOC(CH₂)_{z}NH₂ |
| (Examples) | | | |
| Example | [—O(CₓH_{(2x+2-y)})O(CO)—(CᵤH₂ᵤ)(CO)—]ₙ | [—O(CₓH₂ₓ)O(CO)—NH((C₆H₃)CH₃)NH—]ₙ | [—NH(C_{y}H_{2y})NH(CO)—(CᵤH₂ᵤ)(CO)]ₙ |
| Formula(s) | where v = y = 2 | where y = 2 and the Diisocyanate = | or [NH(CH₂)_{z}(CO)]ₙ |
| of Polymer | | TDI | |
| Products | The components used to synthesize | The polyhydric alcohols used to | The same components used to synthesize |
| from Hydro- | the polyester, e.g., polyhydric | synthesize the PU, and the diamines | the polyamide |
| chemolysis | alcohols and di- and tri-functional | corresponding to the diisocyantes | |
| | carboxylic acids | used to synthesize the PU | |

The diversity of MM1/synth in the feedstock determines the complexity of Product1 obtained from Reactions1. The simplest case is illustrated in embodiments where the MM1/synth is a particular nylon-e, A1 is water, and Reactions1 substantially yield HOOC(CH₂)ₑ₋₁NH₂ as the product, which corresponds to the aminocarboxylic acid from which the nylon-e was produced, or to the lactam from which it was produced. Next simplest is when MM1 is a particular nylon-c,d, the product now being substantially an equimolar mixture of the diamine H₂N(CH₂)_{c}NH₂ and the diacid HOOC(CH₂)_{d-2}COOH. Similarly, when the macromolecule feedstock is PET, Reactions1 yield an equimolar mixture of EG and terephthalic acid. PET and nylons of all types are representative of thermoplastics, a category of polymers that commonly are produced by companies that supply them in pelletized form to other companies that melt and reform them into diverse products. They are chemically simple, being produced substantially from only one monomer, e.g., as in nylon-e, or two monomers, e.g., as in nylon-c,d and PET. Thus, the chemical composition of nylon-6,6 or PET is substantially the same regardless of the manufacturer.

By contrast, when MM1/synth is PU, or comprises or contains polyester resins made with polyhydric alcohols, product mixtures typically are much more complex, and the exact nature of mixture components is usually unknown and largely unknowable. The reason is twofold. Consider PU foams, which commonly are supplied by companies that specialize in formulating proprietary two-part polyurethane systems containing blowing agents, polymerization catalysts, and flame retardants, to meet performance requirements for customers' specific applications. For example, producers of rigid foam-board insulation used in construction or flexible foam slabs used in seat cushions or mattresses do not necessarily produce the pre-polymeric polyols and isocyanates, but purchase them ready to use from system suppliers. Alternatively, large producers of PU foam products may formulate their own. Regardless, PU systems are seldom, if ever, formulated from a single polyhydric alcohol and may even use more than one type of diisocyanate. Consider further that PU recovered from post-consumer material streams inevitably will be of diverse origins, e.g., foams recovered from mattresses, seat cushions, automobiles, and building renovations. The situation may be further aggravated in post-consumer material streams that contain not only PU but also and/or polyamides (nylons) and/or polyesters, the composition of each being diverse and indeterminate.

Accordingly, in particular embodiments that promote Reactions1 in the macromolecule feedstock, (i) the feedstock is MM1/synth comprising or containing one or more taken from the group consisting of but not limited to polyesters, polyester resins, PU, and polyamides; and (ii) A1T1 is water; and (iii) the reaction mixture is obtained by contacting the feedstock with A1T1 at elevated temperature and pressure in the presence of A2; and (iv) the feedstock-A1T1 mass ratio is between about 2:1 and about 1:8; and (v) T(range)1 is between about 200° C. and about 330° C. and the pressure is sufficient to maintain greater than about 15% of A1 in the liquid phase; and (vi) Products1 from MM1/synth include components corresponding to those that were combined to form MM1/synth in the feedstock including but not limited to polyhydric alcohols, polybasic carboxylic acids, and polyamines, as depicted in Table I.

Second Desirable Reactions of Certain MM1/synth. Following the aforementioned deconstruction of MM1/synth by Reactions1, and under conditions associated with Reactions1, additional reactions can occur in cases where the product mixture includes components that comprise a saturated hydrocarbon moiety bearing hydroxyl and/or amine functionality, which was involved in heteroatom linkages whose formation defined the backbone, chain, network, or matrix of certain MM1/synth. The additional reactions cause the in situ elimination of heteroatoms in such functionality, subsequent to formation of the components in the first product mixture through Reactions1, obtaining thereby hydrocarbons as depicted in reactions (4)-(5).

Nonlimiting examples of components that undergo conversion to hydrocarbons are one or more taken from the group including polyhydric alcohols from polyester resins and/or polyurethanes and diamines from polyamides. By contrast, carboxyl functionality in polybasic carboxylic acids from polyesters and/or polyamides does not readily undergo elimination decarbonylation or decarboxylation reactions under the same conditions. Components that are difunctional have the general formula (CᵤHᵥ)X₂, where X=—OH, —NH₂, or —COOH and v has values between about 2u and about 2u−2. When the hydrocarbon moiety (CᵤHᵥ) comprises a quantity n of methylene groups, then the components have the general formula X(CH₂)ₙX, which undergo elimination to obtain a diolefin CH₂═CH(CH₂)ᵤ₋₄CH═CH₂ in accordance with reactions (4)-(5) by dehydration and deaminiation when X=—OH, and —NH₂, respectively. Alkenes are more reactive than saturated hydrocarbons, which makes their presence in the product mixture potentially problematic due to the possibility for them to react with each other under the prevailing conditions of Reactions1. As will be examined hereinbelow, a similar problem arises in connection with second macromolecules.

### Production of Hydrocarbons by Reactions2.

### Dehydration of Alcohols

RCH₂CH₂OH→RCH₂═CH₂+H₂O (4)

### Deamination of Amines

RCH₂CH₂NH₂→RCH₂═CH₂+NH₃ (5)

Promotion of Reactions2. In embodiments where the first product mixture contains polyhydric alcohols and A1T1 is water, the latter will strongly inhibit reaction (4) because water is a product. Accordingly, particular embodiments promote reaction (4) in the first product mixture by substantially removing all liquid water from the product mixture by distillation, which also serves to further drive reaction (4) by removing water produced by it.

Reactions1 of MM1/renew. In other particular embodiments, MM1 are renewable macromolecules, MM1/renew, derived from renewable feedstocks, nonlimiting examples of which include cellulose, lignin, lignocellulose, renewable oils, and biomass. the MM1/renew all comprise molecular substructures linked through heteroatom-containing functionality, nonlimiting examples of which include esters and ethers such as are found in MM1/synth, as well as acetal, hemiacetal, hemiketal, and peptide functionality, all of which are susceptible to deconstruction by Reactions1 according to embodiments described herein.

Feedstock: Second Macromolecules. In other particular embodiments that promote deconstruction of macromolecules through Reactions1, the feedstock includes materials that are second macromolecules (MM2), which, like MM1, also are diverse but differ from MM1 insofar as (i) the linkages that define the polymer backbone, chain, matrix, or network substantially comprise bonds between carbon atoms instead of heteroatoms; and (ii) they are not renewable. Nonlimiting examples of MM2 include: synthetic polymers (MM2/synth) with the general formula (CH₂CRR′)ₙ; higher-MW components of heavy oil and bitumen, e.g., resid (MM2/resid), including but not limited to asphaltenes, and also maltenes whose polarity and/or MW are elevated compared with other maltenes; and the fraction of tire rubber that substantially comprises hydrocarbon polymer (MM2/tire). In the general formula (CH₂CRR′)ₙ for MM2/synth, the two carbon atoms represent the polymer chain or backbone while R and R′ are substituents on the same, nonlimiting examples of which include: (i) R=H and R′=H, methyl, ethyl, vinyl, propyl, isopropyl, butyl, pentyl hexyl, cyclohexyl, phenyl, heptyl, octyl, and the like; (ii) R=R′=methyl; and (iii) R=H and R′=Cl, which is polyvinylchloride. Nonlimiting examples of (CH₂CRR′)ₙ include common synthetic polymers such as polyethylene (PE), polypropylene (PP), and polystyrene (PS) in which R′=H, methyl, and phenyl, respectively, and R=H. In MM2/resid, covalent linkages within and between molecular substructures may involve sulfur, nitrogen, and even metals while the overall structure of the macromolecule is defined substantially by bonds between carbon atoms. In MM2/tire, the hydrocarbon fraction of tire rubber that comprises macromolecules may be thought of as having a primary and secondary structure. Wishing to not be constrained by any particular theory of operation, the former may be regarded as being a polymer chain or backbone, which is typically formulated variously from monomers such as styrene, butadiene, and isoprene, and in some cases from natural rubber such that the primary structure is defined substantially by bonds between carbon atoms; and the secondary structure may be regarded as the matrix or network required to provide the required mechanical and chemical durability, which is created by cross-linking of primary structures and commonly involves heteroatoms, e.g., vulcanization in which the heteroatom is sulfur. Although tire rubber is related to MM1 in respect of heteroatoms that confer secondary structure, it is included with MM2 in respect of its primary structure.

Necessity of Chemical Quenching. In Reactions1 that chemolytically deconstruct MM2, A1 is water, but in contrast with MM1 it is not incorporated into the lower-MW product fragments from MM2. And although reactive alkene-containing lower-MW fragments can be generated from only certain components in product mixtures obtained from MM1, thermochemolysis of MM2 yields a preponderance of one or more types of intermediates that are unstable or metastable and, to varying degrees, reactive. Though wishing to not be constrained by a particular chemical theory, such intermediates may contain reactive functionality in the form of carbanions, carbocations, alkenes, or free radicals. Neutralization or quenching of such anionic and cationic species may occur straightforwardly by well-understood mechanisms, e.g., proton transfer from water to the anion and reaction of the resulting hydroxide ion with the cation to form an alcohol that subsequently dehydrates to form an alkene. Or, the alkene forms directly when a carbanion deprotonates a carbocation. But problematically, alkenes formed by those or other mechanisms can react with carbocations, free radicals, and even with each other; and carbocations can react with carbanions. Such recombinations of reactive molecular fragments can continue to yield macromolecules even larger than those in the macromolecule feedstock. Confronted with this possibility for product mixtures from Reactions1 involving both MM1 and MM2, the inventors recognized the need to prevent such undesirable reactions through the deliberate promotion of third desirable reactions that preserve the benefits of Reactions1 and Reactions2, e.g., the production of lowerer-MW fragments from MM. Accordingly, particular embodiments that deconstruct macromolecules into lower-MW fragments through Reactions1 involve, as appropriate, a third agent (A3) that facilitates the quenching of reactive intermediates including the reduction of alkene functionality.

The Unsuitability of Molecular Hydrogen. The long-established way to accomplish such quenching and reduction is through addition of molecular hydrogen (H₂) through processes that may be referred to generally as hydrotreating. This can be very economical and efficient in certain contexts, e.g., in integrated petroleum refineries. Yet, disadvantages also attach to hydrotreating, which relate to the hydrogen source, reaction conditions, energy requirements, scale, and emissions. One challenge to be overcome in hydrotreating is that H₂ may be regarded as relatively stable, even inert, apart from conditions where it is not, the best examples of the latter being perhaps the Hindenburg and space launch vehicles. But in the context of chemical production processes, hydrogen must be induced to react, which is commonly accomplished through employment of heterogeneous catalysts at elevated partial pressures for hydrogen, e.g., 1000 psi.

A second issue with H₂ is that the most common, practical, and economical way to produce it is by catalytic methane-steam reforming (MSR) for which the net reaction is CH₄+2H₂O→CO₂+4H₂. It is a specific example of processes that produce H₂ from diverse feedstocks with the formula CᵤHᵥ according to the net equation, CᵤHᵥ+2u H₂O→u CO₂+(2u+v/2) H₂. Alternatives to methane (u=1, v=4) include coal (0≤v<<1) and hydrocarbons like naphtha (typically u=5 to 10 and 2u≤v≤2u+2), but the name MSR denotes the paramountcy of natural gas as a feedstock. As implied by the words steam and catalytic, production of H₂ by such processes is energy-intensive and requires catalysts. Inside integrated petrochemical operations where infrastructure requirements can be met relatively straightforwardly, catalytic steam reforming is practical and economical. But it is less so in small-scale, stand-alone implementations due to diminished efficiencies as well as capex and opex considerations. Any view that conventional reforming technology is an efficient means for producing H₂ necessarily ignores the environmental cost of greenhouse gas (GHG) emissions. According to the equations given above, each tonne of hydrogen produced yields between 5.5 tonnes and 11 tonnes of CO₂ when the feedstocks are pure methane and carbon, respectively. But for MSR, a comprehensive accounting of all factors, including energy requirements, yields a more realistic, accepted figure of nine tonnes or more of CO₂ per tonne H₂ produced. Yet, even if so-called blue and green hydrogen eliminates GHG emissions and facile approaches are developed for H₂ production and handling, these do not mitigate the aforementioned conditions for the application of H₂ for the present purposes of chemical quenching.

Chemical Quenching by Third and Fourth Reactions. Due to the many issues with H₂ just enumerated, the inventors determined that the needed third agent should not be H₂. Efforts to find an alternative led to the discovery of an altogether novel and nonobvious phenomenon whereby third desirable reactions (Reactions3) quench the reactive fragments from Reactions1 and/or Reactions2 by operation of hydrogen equivalents [H] generated when a third agent (A3) with the general formula CᵤHᵥO_{w} undergoes fourth desirable reactions (Reactions4). Though wishing to not be bound by any particular theory of operation, such A3 materials are thought to undergo in situ aqueous reforming (AR) in the reaction mixture to generate the aforementioned hydrogen equivalents in analogy with aqueous-phase reforming (APR). The chemical reactions involved in APR are commonly portrayed in the literature as the same, familiar two-step process of steam reforming: reaction between water and some compound CᵤHᵥO_{w} to produce synthesis gas (syngas); and subsequently the water-gas shift reaction (WGSR). Specifically, the products in syngas are given as carbon monoxide (CO) and H₂ while products of the shift reaction are given as CO₂ and H₂. Equations (6)-(8) present an alternate accounting of AR by Reactions4 in embodiments of the instant invention where water may exist in the liquid and/or gas phases. Although CO₂ is a product in accordance with customary representations of APR, Equations (6) and (7) do not adhere to the customary representation of CO as a discrete intermediate that undergoes oxidation to CO₂, but rather as [CO]. Likewise, Equations (6)-(8) do not denote hydrogen as the discrete chemical specie H₂, but as [H].

Production of [H] from A3 Compounds CᵤHᵥO_{w} by Reactions4.

### Syngas Analogy

CᵤHᵥO_{w}+(u−w)H₂O→u[CO]+(2u+v−2w)[H] (6)

### Water-Gas Shift Reaction Analogy

u[CO]+u H₂O→uCO₂+2u[H] (7)

### Net In Situ Aqueous Reforming Reaction

CᵤHᵥO_{w}+(2u−w)H₂O→uCO₂+(4u+v−2w)[H] (8)

Here, the bracketed terms [H] and [CO] denote chemical equivalents, not concentrations and should be understood to mean hydrogen equivalents and CO equivalents, respectively, or alternatively H(equiv) and CO(equiv). This connotes that (i) the specie within the brackets operates within the reactions as if it existed in that discrete form while (ii) it may actually exist in some other chemical form of a plurality of chemical forms, or may be the net result of other reactions not specified or known. Therefore, [H] and [CO] are expressions of stoichiometric or chemical equivalence but do not necessarily denote chemical identity; they represent net outcomes for conversion of reactants CᵤHᵥO_{w} to the products CO₂ and [H] through unspecified chemical mechanisms. Thus, [CO] denotes a chemical reality that may or may not formally involve the discrete compound CO; it is a representation of a reality that may be more complex. Similarly, in the context of Reactions3, [H] operates to provide chemical outcomes substantially equivalent to those that would be obtained as if H₂ were available and could be made to effect the desired quenching, but the chemical form and reactivity of [H] are different from that of H₂; and 2[H] is stoichiometrically equivalent to H₂ but not necessarily formally equivalent. Similar rationale underlies the identification of reactions (6) and (7) as analogous to those associated with syngas production and with WGSR, respectively: the equations denote stoichiometric but not formal equivalence.

Evidence for the production [H] by Reactions3 is well established for feedstock MM of the two kinds, MM1 and MM2. In Case 1, the heteroatom linkages in MM1 are esters and the substructures contain alkene functionality, yet components obtained after MM1 deconstruction by Reactions1 are substantially saturated in the presence of A3 while in its absence the unsaturations persist substantially quantitatively in the product mixture. In Case 2, the MM2 was asphaltenes separated from bitumen. When subjected to conditions that promoted Reactions1 with and without A3, the product from Reactions1 was initially a liquid of relatively low viscosity that flowed freely at ambient T in contrast with the starting material, which was a granular solid. In the absence of A3, the product hardened into an intractable solid within 24 hours at ambient T, evidencing the recombination of reactive or metastable intermediate fragments. By contrast, addition of A3 to the first reaction mixture obtained a product that was altogether stable, persisting indefinitely as a liquid.

In Cases 1 and 2, if A3 were quantitatively converted to H₂ and CO₂ by AR, then the partial pressure of H₂ would have been 79 psi and 1.7 psi, respectively, in the reactors under the conditions employed. But supposing this happened, the partial pressure of H₂ would never reach even those levels, as at least some of it would be consumed in Reactions2. Yet, even the highest value is well below the range that is typical in hydrotreating, e.g., 150 psi-2500 psi, and the reduction occurred in the absence of any heterogeneous catalyst such as those used to promote hydrotreating. These facts point to the generation instead of [H] and its operation in a fashion that obtains products corresponding to the addition of hydrogen but without the intermediacy of H₂. And even if A2 operated catalytically to promote hydrotreating by mechanisms involving H₂, the rates would be so low as to be irrelevant for the reasons just enumerated. This suggests instead that quenching in Reactions3 occurs substantially by chemical mechanisms different from those in conventional hydrotreating, warranting the designation of [H] instead of H₂ on the grounds that the observed results cannot be explained by the generation and operation of the latter.

Third Agent Type 1. In particular embodiments that upgrade MM2 by Reactions1 and Reactions2, A3 comprises compounds with the empirical formula CᵤHᵥO_{w} and includes one or more compounds or mixtures of compounds whose composition relates generally to three types of materials. Third Agent Type 1 (A3T1) is one or more materials taken from the group for which representative empirical formulas include by way of nonlimiting example: (Cₓ(H₂O)_{y})ₙ, which includes carbohydrates (n≥1) and also polysaccharides and cellulose (n>1); (CH₂O)ₙ, which includes monosaccharides, e.g., n=3, 5, or 6; (C₆H₁₀O₅)ₙ or C₁₂H₂₂O₁₁, which also may include cellulose; and CₓH₍₂ₓ₊₂₎O_{y}, which includes alcohols, diols, triols, and tetrols (y=1, 2, 3, and 4, respectively, and x≥y) as well as sorbitol (x=y=6) and sorbitan (x=6, y=5); (CH₂CH(OH))ₙ, which includes polyvinyl alcohol; and (CᵤHᵥO_{w})ₙ where u is between about 28 and about 34 and v is between about 30 and 38 and w is between about 9 and about 13, which includes lignin for which (C₃₁H₃₄O₁₁)ₙ is representative. Nonlimiting examples of diols include: ethane-1,2-diol, propane-1,2-diol, propane-1,3-diol; butane-1,2-diol and its 1,3, 1,4, and 2,3 isomers. Nonlimiting examples of triols include: 1,2,3-propane triol and 1,2,4-butane triol. And nonlimiting examples of tetrols include erythritol and pentaerythritol (x=4 and 5, respectively). These examples illustrate two general characteristics of A3T1: (i) they span a wide range of molecular weights, from relatively low-MW compounds containing between one and six carbon atoms, to polymeric, higher-MW compounds comprising an assemblage of one or several monomeric or monomer-like substructures (e.g. polysaccharides, cellulose, and lignin); and (ii) with the exception of simple alcohols CₓH₍₂ₓ₊₂₎O_{y} (y=1) and lignin, both the low-MW molecular weight compounds and the substructures of high-MW compounds bear a plurality of hydroxyl functionality.

Third Agent Type 2. Third Agent Type 2 (A3T2) includes by way of nonlimiting example one or more materials with the empirical formula RO(CₓH₂ₓO)ₙR, which include polyoxyalkylene dialkyl ethers (R=methyl, ethyl, propyl, butyl, etc.) and the corresponding diols (R=H). Nonlimiting examples of polyoxyalkylenes include: polyoxymethylene, polyoxyethylene, polyoxypropylylene, and polyoxybutylene (x=1, 2, 3, and 4, respectively, and n≥2 with no upper limit for purposes of the instant invention); mixed polyoxyalkylenes made by co-polymerization of two or more alkylene oxides, e.g. of ethylene oxide and propylene oxide such that 2<x<3; and low-MW polyoxyethylene homologs such as diethylene glycol and triethylene glycol (R=H, x=2, and n=2 and 3, respectively), their propylene-based counterparts (x=3), and corresponding mono- and di-alkyl ethers wherein one or both of the R-groups is an alkyl group, e.g., methyl, ethyl propyl, butyl; and polyoxyalkylene adducts of low-MW triols, tetrols, monosaccharides, and sorbitol.

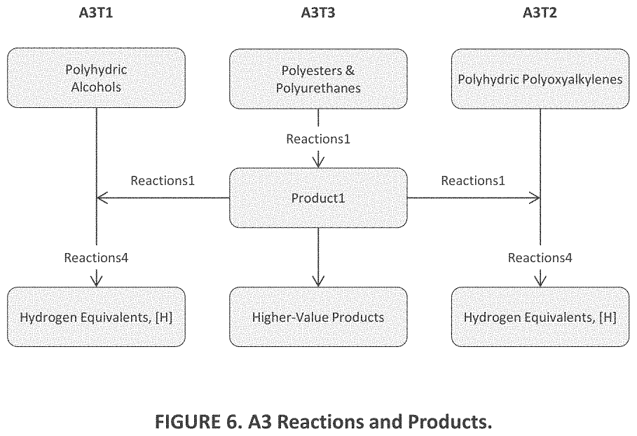

Third Agent Type 3. Third Agent Type 3 (A3T3) includes by way of nonlimiting example polyesters, polyester resins, and polyurethanes produced through reactions with polyhydric alcohols, where nonlimiting examples of the latter include of one or more materials taken from the group consisting of, but not limited to, those with the formulas: CₓH_{(2x+2-y)}(OH)_{y}, which includes diols, triols, and tetrols (y=2, 3, and 4, respectively, and x≥y) as well as sorbitol (x=y=6) and sorbitan (x=6, y=5); (CₓH_{y})(OH)₂, which includes alkylene diols, where y=2x or 2x−2 and alkylene includes by way of nonlimiting example trimethylene, butylene, and cyclohexenedimethylene; HO(CₓH₂ₓO)ₙH, which includes polyoxyalkylene diols; and hydroxyl-terminated polyoxyalkylene adducts of any of the aforementioned diols, triols, or tetrols, or of sorbitan or sorbitol. Nonlimiting examples of polyoxyalkylenes include: polyoxymethylene, polyoxyethylene, polyoxypropylylene, and polyoxybutylene (x=1, 2, 3, and 4, respectively, and n has values from 2 to about 25); mixed polyoxyalkylenes made by co-polymerization of two or more alkylene oxides, e.g. of ethylene oxide and propylene oxide such that 2<x<3; and low-MW polyoxyethylene homologs such as diethylene glycol and triethylene glycol (x=2, and n=2 and 3, respectively), their propylene-based counterparts (x=3). Deconstruction of A3T2 and A3T3. The designations A3T1, A3T2, and A3T3 permit the relating of A3 materials to each other in terms of their composition and the reactions they undergo in particular embodiments to the ultimate end of generating [H]. For example, when A3 consists of or includes A3T3, the ester or urethane linkages are susceptible to hydrolysis under conditions that promote Reactions1, which yields carboxylic acids and amines, respectively, in addition to polyhydric alcohols (Table I). FIG. 6 depicts that the latter may be A3T1 or A3T2, as determined by the composition of the polyesters, polyester resins, or polyurethanes in A3T3. Similarly, those skilled in the art will recognize that the A3T2 are polyethers, and that the ether linkages represent latent hydroxyl functionality due to the possibility for them to undergo hydrolysis. Equation (9) depicts this in customary fashion as being catalyzed by a strong acid, HA. Yet, particular embodiments that promote Reactions1 in MM1/synth also promote hydrochemolysis that depolymerizes A3T2 polyoxyalkylenes as depicted in Equation (10), where A1 is H₂O, A2 is the second agent, the application of heat is denoted by the customary use of the symbol Δ, and the product is an alkylene diol. Thus, when polyesters and polyurethanes containing polyoxyalkylene polyols undergo deconstruction by Reactions1, those polyols in Product1 likewise can undergo deconstruction to obtain alkylene diol products A3T1, which in turn are available to undergo Reactions4 to generate [H] according to Equation (11). Those skilled in the art will recognize the that equations (10) and (11) do not necessarily occur sequentially, but in particular embodiments can occur concurrently, depending on the proximity of T(range)1 to T(range)3. For example, in polyoxyalkylene diols HO(CₓH₂ₓO)ₙH, AR mechanisms may operate directly on the terminal hydroxyl groups at the same time as chemolytic mechanisms operate to progressively hydrolyze ether linkages when T1/max is at least T4/min.

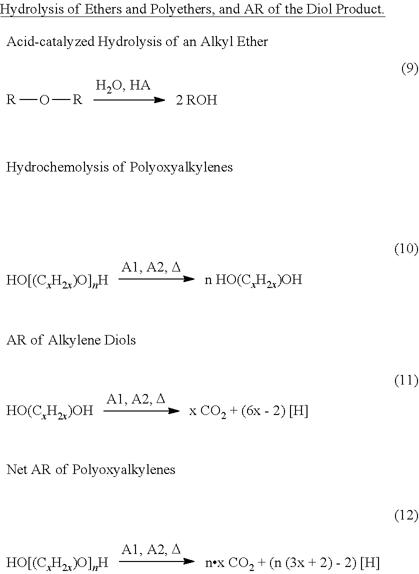

The designations A3T1, A3T2, and A3T3 are indicative. For example, polyoxyalkylene adducts of low-MW polyhydric alcohols are indicated as A3T2 but their complete deconstruction through Reactions1 yields the polyhydric alcohols and alkylene diols, both of which are A3T1. Also, lignin is regarded generally as a phenolic polymer wherein the monomers are substantially derived form phenylpropane. It is designated A3T1 because, first, a significant but indeterminate fraction of oxygen in lignin exists as hydroxyl functionality; and second, lignin and another A3T1 material, cellulose, are closely associated, as denoted in the compound term lignocellulose, which is one of the most abundant forms of fixed carbon in the biosphere. Yet, a considerable fraction of oxygen in lignin exists in the form of ether linkages, making it analogous to the A3T2 polyoxyalkylenes. Thus, the designation of lignin as A3T1 is neither arbitrary nor absolute, nor is it intended to be limiting. Rather, in all cases the purpose of the designations is to organize and illustrate the chemistry that is operative in connection with production of [H] by Reactions4.

Operation of Third Agents. A3 compounds suitable for generating [H] by Reactions4 contain oxygen-bearing carbons whose oxidation numbers are by definition positive. In the examples given for A3T1, A3T2, and A3T3, the oxygen-bearing carbon atoms of predominant importance are those associated with hydroxyl and ether functionalities while acetal, hemiacetal, and hemiketal functional groups also may be present at lower levels in certain A3T1 compounds. By contrast, compounds comprising purely hydrocarbon functionality (CᵤHᵥO_{w} where w=0), wherein carbon atoms are bonded only to other carbon atoms and/or hydrogen, have oxidation numbers that are by definition negative. Such is the case of MM2 wherein the polymer backbone, chain, matrix, or network is defined substantially by carbon atoms bonded to each other and are substantially lacking in heteroatoms, e.g., oxygen atoms are neither interposed between nor appended to carbon atoms in functionalities such as hydroxyl, ether, or ester groups. The susceptibility of such compounds to undergo Reactions4 is significantly lower compared with A3 compounds. Indeed, in particular embodiments of the instant invention that operate on carbon-carbon bonds to deconstruct MM2 through Reactions1 to obtain lower-MW fragments, the latter substantially resist AR, a fact that corresponds to the to absence of oxygenated functionality and low oxidation numbers, e.g., −3 and −2 for methyl and methylene groups in saturated hydrocarbons.

At the other extreme is carboxylate functionality in which the oxygen-bearing carbon atom has, by definition, a high positive oxidation number of +6, e.g., in the carboxylic acid (—COOH) group or in esters (—COOR) thereof, which undergo hydrolysis in particular embodiments to obtain the corresponding carboxylic acid and alcohol ROH. Unlike particular embodiments wherein alcohols and amines undergo Reactions2 as depicted in equations (4) and (5), respectively, or materials A3 undergo Reactions4, carboxylate undergoes elimination by Reactions5, as depicted in equations (13) and (14), but at markedly higher severity.

### Production of Hydrocarbons by Reactions5.

### Decarbonylation of Carboxylic Acids

RCH₂CH₂COOH→RCH₂═CH₂+CO+H₂O (13)

### Decarboxylation of Carboxylic Acids

RCH₂CH₂COOH→RCH₂CH₂H+CO₂ (14)

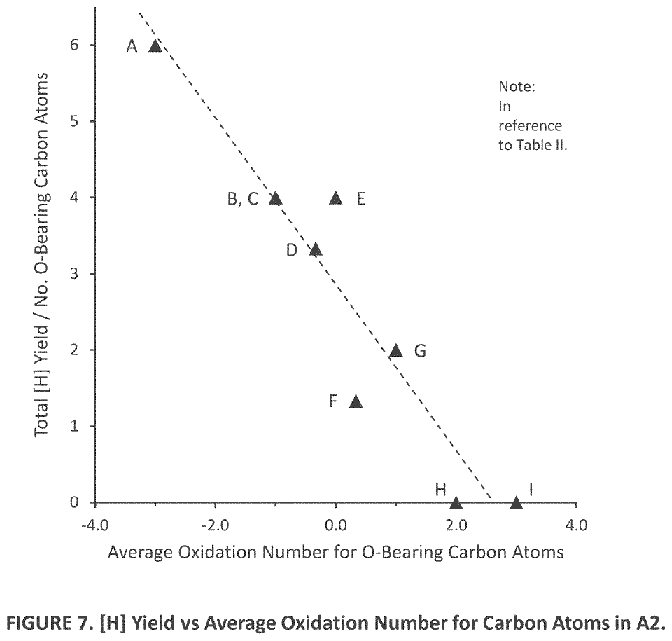

Though wishing to not be constrained by any particular theory of operation, the predisposition, in particular embodiments, of A3 compounds CᵤHᵥO_{w} (w>0) to undergo AR according to Equation (8), and the resulting yield of [H], apparently relates to the oxidation number of oxygen-bearing carbon atoms. By way of nonlimiting example, data for the series of functional groups Table II points to a decline in [H] with increasing average oxidation number, while FIG. 7 shows this general tendency graphically. For example, the oxidation number for the carbon-bearing atoms increases from −1 in B and C to +1 in G while the corresponding yield of [H] is halved. Given that [H] represents equivalents of atomic hydrogen, and that the oxidation state of hydrogen in H₂O is +1, the production of [H] necessarily is the consequence of a redox reaction in which something else is oxidized, i.e., carbon is oxidized to CO₂, whose carbon has the oxidation state of +4. The decrease of [H] yield as a function of increasing oxidation state for the oxygen-bearing carbon(s) relates therefore to diminishment in the change in the oxidation state for oxygen-bearing carbon atoms between reactant and the product, CO₂. Concerning the example reactions given in Table II, those skilled in the art will readily recognize that they (i) are intended to illustrate the dependence of AR outcomes in respect of functional groups with oxygen-bearing carbon atoms in different oxidation states; (ii) are nonlimiting in respect of the balance of example molecules, represented by the R group; and (iii) for clarity do not contemplate AR outcomes for R groups, which may be diverse in A3 compounds of particular embodiments. But in consideration of the vertical axis of FIG. 7, compounds A3 that undergo Reactions4 in particular embodiments preferably contain functional groups for which [H] yields are between about 1 and about 4, where [H] yield is defined as total [H] produced, when the functional group is consumed by AR, divided by the total number of oxygen-bearing carbon atoms in the functional group. That range corresponds to an average oxidation number for oxygen-bearing carbon atoms in compounds A3 of between about −1 and about 1.

Equations (15)-(17) further illustrate the tendency evident in FIG. 7, whereby the yield of [H] from an oxygen-bearing carbon atom decreases as its oxidation increases. Equation (15) also presents an alternative to the fate of alcohols RCH₂OH, shown in Equation (4) as instead undergoing dehydration in Reactions2. This is not a contradiction, but an indication that different outcomes are obtained as a function of (i) the temperature regime, and (ii) the chemical composition of the alcohol. In some A3 compounds CᵤHᵥO_{w} described for particular embodiments, the value of u may be relatively large compared with the value of w, such that in the fraction f of carbon atoms u that are oxygen-bearing is relatively small, where 0<f≤1 and 1−f is the fraction of carbon atoms in CᵤHᵥO_{w} that are not oxygen-bearing. Though wishing to not be limited by any particular theory of operation, the fraction 1−f may not be as susceptible to undergo AR reactions as carbon atoms associated with functionality that does contain oxygen. Equations (15)-(17) illustrate this for the nonlimiting examples of an alcohol, an aldehyde, and a carboxylic acid corresponding to B, G, and I in Table III, respectively, where the R-group for each is a saturated alkyl group (Cᵤ₋₁H₂ᵤ₊₁) and the products in each case are the saturated compound Cᵤ₋₁H₂ᵤ, CO₂, and [H] except in the case of carboxylic acids. Thus, at or below some value of f, portions of compounds CᵤHᵥO_{w} in particular embodiments are not be consumed altogether through AR but instead yield hydrocarbons that substantially contain no oxygen.

TABLE II
Variation in [H] Yield vs Oxidation State of Oxygen-Bearing Carbon.

| | Compound | Avg. | [H] | |
|---|---|---|---|---|
| Compound* | Type | Ox. No.^{†} | Yield ^{‡} | Net AR Reaction^{§} |
| A. RCH₃ | Alkane | −3 | 6** | A + H₂O [symbol] RH + CO₂ 6 [H] |
| B. RCH₂OH | Alcohol | −1 | 4 | B + H₂O → RH + CO₂ + 4 [H] |
| C. RCH₂OCH₂R | Ether | −1 | 4 | C + 3 H₂O → 2 RH + 2 CO₂ + 8 [H] |
| D. RCH(OCH₂R)₂ | Acetal | −0.33 | 3.33 | D + 4 H₂O → 3 RH + 3 CO₂ + 10 [H] |
| E. RCH(OH)(OCH₂R) | Hemiacetal | 0 | 4 | E + 2 H₂O → 2 RH + 2 CO₂ + 8 [H] |
| F. RC(OH)(OCH₂R)₂ | Hemiketal | +0.33 | 1.3 | F + 2 H₂O → 3 RH + 2 CO₂ + 4 [H] |
| G. R(CO)H | Aldehyde | +1 | 2 | G + H₂O → RH + CO₂ + 2 [H] |
| H. R(CO)R | Ketone | +2 | 0 | H + H₂O → 2 RH + CO₂ + 0 [H] |
| I. R(CO)OH | Carboxylic | +3 | 0** | I → RH + CO₂ + 0 [H] |
| | Acid | | | |

*R represents the rest of the organic molecule and the parentheses around CO are included to emphasize that it is a carbonyl group in which the carbon-oxygen bond is a double bond, C═O. Although R is the same in the examples A-I, this should not be understood to connote that all R-groups are necessarily the same nor that no functionality in them is capable of undergoing Reactions4.
^{†}Average oxidation state for all oxygen-bearing carbon (underscored) in the functional group.
^{‡} Total [H] produced, when the functional group is consumed by AR, divided by the total number of oxygen-bearing carbon atoms in the functional group.
**Values for [H] yield correspond to those that would apply if the functional group were to be consumed through Reactions4. (Provided for comparison only. The methyl group contains no oxygen, and both it and the carboxyl group are substantially unsusceptible to Reactions4.)
^{§}In embodiments that promote third desirable reactions.

AR of Terminal Oxygenated Functional Groups in Saturated Compounds CᵤHᵥO_{w}.

### Saturated Alcohol

(Cᵤ₋₁H₂ᵤ₋₁)CH₂OH+H₂O→Cᵤ₋₁H₂ᵤ+CO₂+4[H] (15)

### Saturated Aldehyde

(Cᵤ₋₁H₂ᵤ₋₁)CHO+H₂O→Cᵤ₋₁H₂ᵤ+CO₂+2[H] (16)

### Saturated Carboxylic Acid

(Cᵤ₋₁H₂ᵤ₋₁)COOH→Cᵤ₋₁H₂ᵤ+CO₂+0[H] (17)

Table II, the discussion associated with it, and EA1T1quation (17) also establish that when A3T1 compounds CᵤHᵥO_{w} contain oxygen not only in hydroxyl groups, but also carboxylic acid and ketone functionalities, then the latter yield CO₂ without the beneficial production of [H] in particular embodiments that promote Reactions4. Thus, when CᵤHᵥO_{w} contains the quantity “a” of carboxylic acid or ketone equivalents, the formula C₍ᵤ₋ₐ₎HᵥO_{(w-2a)} helpfully denotes the portion of CᵤHᵥO_{w} that is available to function as [H]-producing A3 and points to the requirement that w>2a. In particular embodiments employing A3, (i) CᵤHᵥO_{w} represents the formula of a specific compound and/or the aggregated composition for a plurality of compounds that together comprise A3; (ii) oxygen atoms are present in one or more functional groups taken from the group consisting of hydroxyl, ether, aldehyde, acetal, and hemiacetal functionalities; (iii) oxygen atoms may additionally be present in carboxylic acid and/or ketone functionality provided w>2a; (iv) the ratio (w−2a)/(u−a) is between about 0.1 and about 1. In particular embodiments, A3 consists of A3T1 for which (w−2a)/(u−a) is between about 0.25 and about 1 while [H] yields are even more favorable when that ratio is between about 0.5 and about 1 and are more favorable still when the ratio is between about 0.75 and about 1. In other particular embodiments, A3 consists of A3T2 lacking in substantial quantities of carboxylic acid functionality such that a<<w, and values for w/u are between about 0.5 and about 2 while [H] yields are even more favorable when that ratio is between about 0.5 and about 1 and are more favorable still when it is between about 0.7 and about 1.

Table III presents an alternative characterization of [H] yield for compounds A3 in particular embodiments, it now being defined as [H] obtained per mole carbon in those compounds according to Equation (8). Accordingly, [H]/C values in particular embodiments are between about 4 and about 6 when A3 compounds include A3T1 and A3T2 except for lignin, where A3T1 and A3T2 include compounds obtained from A3T3 by Reactions1.

TABLE III
Hydrogen Equivalent Yield for A3T1 and
A3T2, [H] per Mole Carbon.

| Second Agent* | | Formula | x | y | [H]/C |
|---|---|---|---|---|---|
| Polysaccharides, | | (Cₓ(H₂O)_{y})ₙ | 6 | 6 | 4.00 |
| Carbohydrates, etc. | | | | | |
| Cellulose | | (Cₓ(H₂O)_{y})ₙ | 6 | 5 | 4.00 |
| Monosaccharides | | (CₓH_{y}O)ₙ | 1 | 2 | 4.00 |
| Sorbitol (x = y = 6) | | CₓH(₂ₓ₊₂)O_{y} | 6 | 6 | 4.33 |
| Sorbitan (x = 6, y = 5) | | CₓH(₂ₓ₊₂)O_{y} | 6 | 5 | 4.67 |
| Alcohols (y = 1) | | CₓH(₂ₓ₊₂)O_{y} | 1 | 1 | 6.00 |
| | | | 2 | 1 | 6.00 |
| | | | 3 | 1 | 6.00 |
| | | | 4 | 1 | 6.00 |
| Triols | glycerol | CₓH(₂ₓ₊₂)O_{y} | 3 | 3 | 4.67 |
| | butanetriol | | 4 | 3 | 5.00 |
| Tetrols | erythritol | | 4 | 4 | 4.50 |
| | pentaerythritol | | 5 | 4 | 4.80 |

| | | | | |
|---|---|---|---|---|
| Polyvinyl alcohol | (CₓH₂ₓO_{y})ₙ | 2 | 4 | 5.00 |
| Oxyalkylene diols and | HO(CₓH₂ₓO_{y})ₙH | 1 | 2 | 4.00 |
| Poly(oxyalkylene) | | 2 | 2 | 5.00 |
| diols (A3T2) | | 3 | 2 | 5.33 |
| | | 4 | 2 | 5.50 |

*All examples are A3T1 except as noted.

Operation of Second Agent (A2). Mix1, the first reaction mixture, is constituted through the optional combining of the prepared feedstock with A1T2 to obtain a premixture, and the further combining of feedstock or the premixture, as the case may be, with A1T1 and A2. Already considered is the possibility that A2 might serve a catalytic role to promote both the production of H₂ by mechanisms other than those underlying Reactions4 to achieve outcomes such as those from Reactions3, in analogy with conventional hydrotreating. However, for reasons discussed, which also are obvious to those skilled in the art, this is unlikely and its relevance is highly limited, to the extent it happens at all. Instead, outcomes from particular embodiments that promote Reactions1-Reactions5 suggest that individually and in aggregate, the reactions operate by mechanisms novel and nonobvious. Table IV presents summary descriptions of the reactions and their important outcomes while Table V provides additional detail concerning their realization in various embodiments. Both tables also introduce Reactions6 that are not chemolytic reactions but operate to further improve the liquid yield obtained when MM are residua.

TABLE IV
Description and Summary of Important Outcomes for Reactions1-Reactions6.

| | Description | Outcome |
|---|---|---|
| Reactions1 | Chemolytic deconstruction of macromolecules | Formation of lower-MW |
| | MM by operation of first and second agents in a | fragments that, in the case of |
| | reaction mixture (Mix1) to obtain a first product | certain feedstock MM, may |
| | mixture (Product1) | contain reactive functionality |
| | | capable of undergoing undesirable |
| | | reactions whereby the fragments |
| | | recombine |
| Reactions2 | Elimination of heteroatom-containing functional | Formation of alkenes, which are |
| | groups in certain MM fragments in Product1, | reactive functionalities that may |
| | e.g., alcohols and amines to obtain a second | undergo the undesirable reactions |
| | product mixture (Product2) | |
| Reactions3 | Quenching of reactive functionalities in MM | Prevention of undesirable reactions |
| | fragments in Product2 by hydrogen equivalents | by substantially eliminating |
| | [H] to obtain a third product mixture (Product3) | reactive functionalities |
| Reactions4 | In situ aqueous reforming of third agents | Production of hydrogen |
| | | equivalents [H] required in |
| | | Reactions3 |
| Reactions5 | Elimination of carboxyl functionality from | Production of saturated |
| | lower-MW fragments of certain feedstock MM | hydrocarbon moieties in MM |
| | and the further operation of Reactions3 and | fragments |
| | Reactions4 | |
| Reactions6 | High-severity thermolytic deconstruction of | Increase the liquid yield obtained |
| | residue from Reactions5 when MM is residua | from residua |

TABLE V
Description and Summary of Important Outcomes for Reactions1-Reactions6.

| | | | Reactions3 | | |
|---|---|---|---|---|---|
| Parameter | Reactions1 | Reactions2 | & Reactions4 | Reactions5 | Reactions6 |
| Applicability ^{a} | All MM1 and | For particular | For particular | For particular | For residua |
| | MM2 | MM1 | MM1 and MM2 | MM1 and MM2 | |
| Input | Prepared | Product1 | Product2 | Product3 | Product5 |
| | Feedstock | | | | |
| Output | Product 1 | Product2 | Product3 | Product5 | Product6 |
| A1T1^{b} | Required | In Product1 | In Product2 | In Product3 | In Product5 |
| A1T2 | As Appropriate | | | | |
| A2 | Required | In Product1 | In Product2 | In Product3 | In Product5 |
| A3 ^{c} | | | Required | Required | Required |
| T(range) | T(range)1 | T(range)2 | T(range)3 | T(range)5 | T(range)6 |
| T(range) | T1/min | T2/min ≥ | T3/min ≥ | T5/min ≥ | T6/mi ≥ |
| minimum | | T1/min | T2/min | T3/min | T5/min |
| T(range) | T1/max | T2/max ≥ | T3/max ≥ | T5/max ≥ | T6/max ≥ |
| maximum | | T1/max | T2/max | T3/max | T5/max |
| T(range) for | About 225° C. to | | | About 325° C. to | Not applicable |
| MM1^{d} | about 370° C. | | | about 370° C. | |
| T(range) for | About 325° C. to | | | About 325° C. to | About 370° C. to |
| MM2^{d} | about 370° C. | | | about 370° C. | about 395° C. |
| Time, t | t1 | t2 | t3 | t5 | t6 |
| Range for t | About 2 to | About 2 to | About 2 to | About 2 to | About 2 to |
| (minutes) | about 250 | about 100 | about 250 | about 150 | about 150 |

^{a} Reactions1 operate on all MM while the applicability of, and requirement for Reactions2-Reactions5 are determined by the chemistry of the MM.
^{b}A1T2 is added optionally to the prepared feedstock to promote the disaggregation of MM therein and thereby predispose them to undergo Reactions1.
^{c} Reactions1 may obtain A3 from certain MM1. If the prepared feedstock also contains other MM1 that do not yield A3, and/or contains MM2, then A3 obtained by Reactions1 of the certain MM1 are available for Reactions3/4. Alternatively, A3 from outside sources can be added.
^{d} Values indicated for T/min and T/max are indicative of those which are applicable in respect of MM1 and MM2 and the diversity of chemistries represented within each kind.

Table V shows that all embodiments share a common starting point, which is the deconstruction of MM in the prepared feedstock through the operation of Reactions1, where Reactions1 are enabled through A1T1 and A2 in T(range)1, and where T(range)1 is selected in respect of the susceptibility of the MM to undergo Reactions1. Subsequently, the fate of lower-MW fragments in Product1 spans a range of possibilities in accordance with the chemical nature of the MM and fragments derived from them, which in turn determines possibilities for producing from them higher-value oils, chemicals, or fuels. Possibilities for further processing of Product1 have been detailed hereinabove and summarized in Tables IV and V. Of particular significance is the fact that the A1T1 and A2 persist through any and all processing steps subsequent to Reactions1, e.g., in Product1-Product5. Not wishing to be bound by any particular theory of operation, the activity of those agents is thought to not be only solitary but synergistic, operating separately and concomitantly to enable Reactions2-Reactions5 through a variety of mechanisms. Also, Reactions1 to Reactions5 occur in nominal correspondence with temperature increases from T(range)1 to T(range)5. For example, they may occur somewhat or substantially sequentially in particular embodiments where T(range)1<T(range)2<T(range)3<T(range)5, e.g., the minimum and maximum temperatures in a given temperature range are higher than those of the preceding temperature range. Alternatively, the reactions occur substantially concurrently in particular embodiments where T(range)1 to T(range)5 are about the same, e.g., about 325° C. to about 375° C. for MM1 and about 340° C. to about 375° C. for MM2.

Table V also introduces Reactions6, which apply when MM2 are residua. As has been explained, chemolytic mechanisms for molecular deconstruction have diminished importance in the higher temperature regime of T(range)6 and thermolytic mechanisms dominate. Yet, particular embodiments that promote Reactions6 benefit from the operation of both A2 and A3: A2 still serves a catalytic role, promoting the deconstruction of resid substructures that resist chemolysis at lower severity; and A3 serves to quench reactive functionality of the resulting MM fragments produced at the higher-severity conditions. Reactions6 effectively operate on what may be described as the residue of the residua, which is a high-viscosity liquid infused with A2 that mediates the in situ Reactions4 to generate reducing equivalents by agency of water in the vapor phase.

A plurality of beneficial changes in A1T1 properties at elevated temperatures have been noted already in connection with Reactions1. Similarly, A2 is thought to operate in one or more of a plurality of possible modalities, of which the following four examples are offered by way of illustration without any intention or desire to be limiting or binding as regards theory of operation. First, and conventionally, a metal ion Mᵢ^{+m} in compounds (Mᵢ)ₐX_{b} can function as a Lewis acid capable of associating with electron-rich functionality, e.g., with electron lone pairs in oxygen and nitrogen atoms within certain functional groups in a given MM or in some A3. The association serves to shift the charge distribution in the functional group, creating thereby a corresponding reduction in electron density elsewhere. This produces multiple effects including, making functional groups in MM1 susceptible to nucleophilic attack by A1T1, which directly results in MM deconstruction by Reactions1; promoting dehydration or deamination in Reactions2; and promoting deoxygenation in Reactions5. In all these examples, Mᵢ^{+m} functions as a simple catalyst. The second mode is similar to the first, but instead of associating with the MM substrate, Mᵢ^{+m} forms a complex with A1T1, the net effect being to enhance its pKₐ, which serves to increase the concentration of both hydrogen ions (decrease pH) and the conjugate base, both of which may enhance rates of Reactions1-Reactions5. This mode can be interpreted in accordance with hard-soft acid-base theory (HSAB) which favors interaction between hard hydroxyl groups of A1T1 and softer metals Mᵢ^{+m}.

Third modes of A2 operation are relevant in embodiments where the macromolecules are MM2 and Reactions1 that produce lower-MW fragments occur by scission of carbon-carbon bonds (C—C). In particular embodiments where feedstock macromolecules are MM2/synth and MM2/resid, the extent and rates of such scission are appreciable when the first reaction mixture is heated to T(range)1, but they are not when A2 is withheld from the first reaction mixture. Indeed, in the absence of A2, Reactions1 are substantially inoperative. This points to the role of A2 as catalyst when it is dissolved or suspended in the reaction mixture, which may be explained in terms of Frontier Molecular Orbital theory familiar to those skilled in the art, whereby the highest-occupied molecular orbital (HOMO) of C—C in MM2 interacts with the lowest-occupied molecular orbital (LUMO) of the metal in (Mᵢ)ₐX_{b}, or vice versa so as to lower the energy of intermediates that yield incipient fragments.

In the fourth mode, Mᵢ^{+m} again functions as a catalyst, but in contrast with the first three modes, it participates in oxidation-reduction, serving as a vehicle to transfer reducing equivalents from A3 to reactive functionality in MM fragments, e.g., in Product1 or Product2. In this mode, Mᵢ^{+m} facilitates Reactions3 and Reactions4, which are redox reactions. Cursory consideration of equations (6), (7), (8), (11), (12), (15), and (16), and of the equations in Table II, reveals that they all involve oxidation and reduction: oxidation of oxygen-bearing carbon atoms in A3 to CO₂, in which the oxidation number is +4; corresponding reduction of hydrogen to generate [H]; and reduction by the latter to quench reactive functionalities, e.g., the quenching of radicals and alkenes in Product1, alkenes in Product2, and alkenes formed through decarbonylation in Reactions5.

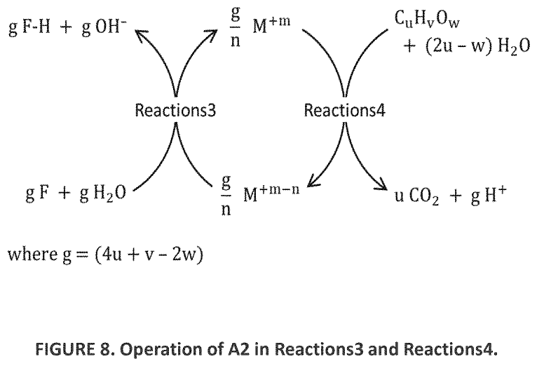

FIG. 8 illustrates a scheme wherein Mᵢ^{+m} serves to transfer reducing equivalents from A3 compounds CᵤHᵥO_{w} to MM fragments (F) containing such reactive functionality. The figure incorporates a modification to Equation (8) wherein the products of Reactions4 include H⁺ instead of [H] while CO₂ is common to both. The outcome is analogous to that obtained in hydrotreating but without the involvement of either H₂ or atomic hydrogen (H): the reduction of F to F—H is mediated by A2, which transfers reducing equivalents from A2 to F. Equation (18) gives the net reaction depicted in FIG. 8.

### Net Quenching of Reactive Fragments F by Reactions3 and Reactions4.

CᵤHᵥO_{w}+(2u−w)H₂O+gF→uCO₂+gF—H (18)

where g=(4u+v+2w)

In this scheme the availability in metals Mᵢ of two or more oxidation states, e.g., Mᵢ^{+m} and Mᵢ⁺⁻ⁿ, is central to the operation of A2. Two possibilities are: m≠n and m>n; and m=n corresponding to the metal in its elemental form. In both cases, the metal may remain suspended in the reaction mixture or the product mixture, as the case may be, and is available to participate in the redox cycle depicted in FIG. 8. Table VI shows common and known positive oxidation states for transition metals in periods 4 and 5 of groups 3-12, as well as for aluminum (group 13, period 3) and tin (group 14, period 5). All have a minimum of three positive oxidation states, with the exception of zinc and cadmium, which only have two. The possibility exists that for a single metal Mᵢ, the Gibbs Free Energy ΔG corresponding to the difference in the electrode potential between the oxidation states Mᵢ^{+m} and Mᵢ^{+m-n} will correspond favorably with ΔG for Reactions3 and Reactions4. That is, ΔG=−n·F·E° for the half reaction corresponding to the change in oxidation state of the metal from +m to +m−n is neither much larger nor much smaller than ΔG for Reactions3 and Reactions4. But to the extent that is not the case, then the mismatch may present a thermodynamic barrier to the overall reaction given by Equation (18). To avoid this limitation and relieve the dependency on a fortuitous free energy matchup, A2 in particular embodiments contains a plurality of metals Mᵢ whose plurality of oxidation states serves to create a “redox ladder” with small-increment oxidation-reduction steps between standard reduction potentials. By way of nonlimiting example, the pairing of vanadium and nickel in a molar-basis ratio of between about 2:1 and 1:1 is efficacious for purposes of enabling Reactions1-Reactions5, where that range in V:Ni corresponds to the relative abundance of those metals in certain heavy oils. And in other particular embodiments, A2 consists of metals of a first and second type, and optionally a metal of a third type, e.g., Metals1, Metals2, and Metals3, where the aggregated molar-basis concentration ratio Metal1:Metal2 is between about 1:4 and about 4:1 and (Metals1+Metals2):Metals3 is between about 2:1 and 10:1.

TABLE VI
Oxidation States for Exemplary A2 Metals Mᵢ^{+m} from Groups 3-14.

| Group | Element ^{a} | Period | Oxidation States ^{c} | | Element ^{a} | Period | Oxidation States ^{c} | |
|---|---|---|---|---|---|---|---|---|
| 3 | Sc | 4 | 3 | (2, 1) | Y ^{b} | 5 | 3 | (2, 1) |
| 4 | Ti ^{b} | 4 | 4 | (3, 2, 1) | Zr | 5 | 4 | (3, 2, 1) |
| 5 | V ^{b} | 4 | 5, 4, 3 | (2, 1) | Nb | 5 | 5 | (4, 3, 2, 1) |
| 6 | Cr | 4 | 6, 3, 2 | (5, 4, 1) | Mo ^{b} | 5 | 6, 4 | (5, 3, 2, 1) |
| 7 | Mn ^{b} | 4 | 7, 4, 2 | (6, 5, 3, 1) | Tc | 5 | 7, 4 | (6, 5, 3, 2, 1) |
| 8 | Fe ^{b} | 4 | 3, 2 | (6, 5, 4, 1) | Ru | 5 | 4, 3 | (8, 7, 6, 5, 2, 1) |
| 9 | Co ^{b} | 4 | 3, 2 | (5, 4, 1) | Rh | 5 | 3 | (6, 5, 4, 2, 1) |
| 10 | Ni ^{b} | 4 | 2 | (4, 3, 1) | Pd | 5 | 4, 2 | (6, 5, 3, 1) |
| 11 | Cu ^{b} | 4 | 2, 1 | (4, 3) | Ag | 5 | 1 | (4, 3, 2) |
| 12 | Zn ^{b} | 4 | 2 | (1) | Cd | 5 | 2 | (1) |
| 13 | Al ^{b} | 3 | 3 | (2, 1) | | | | |
| 14 | | | | | Sn ^{b} | 5 | 4, 2 | (3, 1) |

^{a} All are transition metals except for Al and Sn, which are basic metals.
^{b} Found in crude oils in non-trace levels (typical maximum concentration > 50 ppm).
^{c} Common oxidation states are given first (other known oxidation states given in parentheses).
Negative oxidation states also are possible; their exclusion does not mean they cannot participate in Reactions3 and Reactions4 as depicted in FIG. 8. Positive values in Table VI are illustrative.

Sources of A2. In particular embodiments, A2 is from the aqueous phase isolated by liquid-liquid separation of Product1 obtained by Reactions1 when the prepared feedstock is MM2/resid. And in other particular embodiments, A2 is from a byproduct waste stream generated during production of organotin compounds used as stabilizers in rigid vinyl compounds including house siding, window frames, and PVC piping, in which byproduct the levels of tin are relatively high and those of iron are moderate.

Deoxygenation by Reactions5. Reactions1 operate on ester functionality in certain MM1 to obtain fragments with corresponding hydroxyl and carboxylic acid functionality while carboxylate functionality typically present in MM2/resid already exists in the acid form, e.g., naphthenic acids, which are problematic in downstream refining operations. In both cases, such carboxylic acid functionality is substantially eliminated in the last of the desirable reactions promoted by particular embodiments of the instant invention (Reactions5). For certain MM1 wherein carboxyl-bearing MM fractions contain fewer than about 25 carbon atoms and the carboxyl group is not bonded to an aromatic group, Product5 contains hydrocarbons that are substantially saturated and may be isolated for use as fuels. When feedstock MM are MM2/resid, Product5 contains resid fragments in which the naphthenic acids have been converted to saturated naphthenes.

### Exemplary Embodiments

Reactor. Those skilled in the art will recognize the possibility to realize outcomes from Reactions1 to Reactions6 through a variety of common apparatus implemented according to common practices, including but not limited to batch reactors, semi-continuous reactors, continuous-flow reactors, e.g., shell-in-tube reactors, and combinations thereof, where the significance and meaning of those terms is commonly understood. In particular embodiments that employ batch reactors, temperature adjustment and addition of agents A1, A2, and A3 are performed at such times as are appropriate to promote reactions for MM in the prepared feedstock. The same is done in exemplary embodiments that employ semi-continuous and continuous flow schemes in which flow rate is an additional variable that relates to time while temperature is increased along the flowpath as appropriate to promote Reactions1-Reactions6, as the case may be. The reactions occur substantially concurrently in a single reactor or a plurality of reactor subsections communicably coupled in series and operating in a single temperature range suitable to promote all reactions of interest. In other particular embodiments the reactor is a plurality of reactor subsections communicably coupled in series and configured to operate in successively higher temperature ranges suitable to progressively promote successive reactions in product mixtures obtained from each reaction, e.g., two or more reactor subsections operate at different temperature ranges, each being suitable to promote at least one reaction of interest.

Process Conditions. All embodiments promote outcomes, as desired, which correspond to those from Reactions1 and optionally from Reactions2-Reactions6, as the case may be, by configuring temperature, time, and the amounts of A1, A2, and A3 relative to MM as appropriate, where the time required to promote the reactions is determined in respect of both temperature and the characteristics of MM and fragments derived from them, which determine their susceptibility to undergo the reactions; and where A3 quantities are supplied in accordance with demand defined by quantities of reactive functionality in Product1 or Product2, or in Product6 from Reactions6, as the case may be.

MM in Prepared Feedstock. The foregoing discussion about embodiments of the instant invention is made with a view toward the particular characteristics of various MM1 and MM2 including their susceptibility to undergo initial deconstruction by Reactions1 and also the applicability of Reactions2-Reactions6 in respect of products obtained by Reactions1-Reactions5. That discussion points to the possibility to co-process different MM types. For example, in particular embodiments, the diverse MM identified as MM1/synth or those identified as MM2 may be combined in a given prepared feedstock. In a nonlimiting example of the latter, MM2/tire and/or MM2/synth are combined with MM2/resid. And in a particularly advantageous embodiments, the mixed prepared feedstock contains one or more of MM2/synth, MM2/tire, and MM2/resid together with MM1 including by way of nonlimiting example one or more taken from the group consisting of cellulose; lignin; lignocellulose; post-consumer PU foam; glycerol byproduct from conversion of renewable oils to biodiesel; and ethylene glycol byproduct of PET depolymerization. The benefit of such embodiments resides is the concurrent deconstruction by Reactions1 of MM1 and MM2 materials in the feedstock followed by in situ generation of hydrogen equivalents from MM1 fragments and/or glycerol and/or EG.

Certain aspects of the present invention include process steps and instructions described herein in the form of an algorithm. It should be noted that the process steps and instructions of the present invention could be embodied in software, firmware or hardware, and when embodied in software, could be downloaded to reside on and be operated from different platforms used by real time network operating systems. Moreover, the particular naming of the components, capitalization of terms, the attributes, data structures, or any other programming or structural aspect is not mandatory or significant, and the mechanisms that implement the invention or its features may have different names, formats, or protocols.

Modifications, additions, or omissions may be made to the systems, apparatuses, and methods described herein without departing from the scope of the disclosure. For example, the components of the systems and apparatuses may be integrated or separated. Moreover, the operations of the systems and apparatuses disclosed herein may be performed by more, fewer, or other components and the methods described may include more, fewer, or other steps. Additionally, steps may be performed in any suitable order. It should be further understood that any of the features described with respect to one of the embodiments described herein may be similarly applied to any of the other embodiments described herein without departing from the scope of the present invention. As used in this document, “each” refers to each member of a set or each member of a subset of a set.

To aid the Patent Office and any readers of any patent issued on this application in interpreting the claims appended hereto, applicants wish to note that they do not intend any of the appended claims or claim elements to invoke 35 U.S.C. 112(f) unless the words “means for” or “step for” are explicitly used in the particular claim.

Finally, it should be noted that the language used in the specification has been principally selected for readability and instructional purposes, and may not have been selected to delineate or circumscribe the inventive subject matter. Accordingly, the disclosure of the present invention is intended to be illustrative, but not limiting, of the scope of the invention, which is set forth in the following claims. It should be further understood that any of the features described with respect to one of the embodiments described herein may be similarly applied to any of the other embodiments described herein without departing from the scope of the present invention.

Having thus described the invention, what is claimed is:

1. A method for deconstructing macromolecules (MM) into lower molecular weight (MW) fragments in high yield by promoting first desirable reactions (Reactions1) that result in chemolytic scission of bonds in the backbone, chain, matrix, or network that defines the MM and obtain a first product mixture (Product1), the method comprising:
(a) configuring a flowpath to receive a prepared feedstock containing MM;
(b) conveying the prepared feedstock in a flowpath toward a reactor;
(c) contacting the prepared feedstock in the flowpath or in the reactor with a first agent of a first type (A1T1) suitable for promoting Reactions1;
(d) contacting the prepared feedstock in the flowpath or in the reactor with a second agent (A2) suitable for promoting Reactions1 to obtain a first reaction mixture;
(e) configuring the reactor to heat the first reaction mixture;
(f) heating the first reaction mixture in the reactor to a temperature range T(range)1 in the range between T1/min and T1/max for a length of time t1 to obtain a product mixture Product1;
(g) selecting A1T1 and T(range)1 in respect of MM chemistry and kind, which determines MM susceptibility to undergo Reactions1; and
(h) selecting the total amount of A1T in the first reaction mixture and configuring the reactor to control the total pressure therein to establish amounts of A1T1 that exist in the liquid and gas phases when the first reaction mixture is heated in the reactor to T(range)1, where the amounts of A1T1 in the two phases are selected in respect of MM chemistry and kind and are appropriate to support Reactions1.

2. The method of claim 1, wherein said contacting (d) further comprises configuring A2 in the form of one or more metals Mi in compounds with the general formula (Mi)aXb.

3. The method of claim 2, further comprising selecting the one or more metals Mi from the group consisting of periodic table of chemical elements groups 3-14.

4. The method of claim 3, further comprising selecting the one or more metals Mi from the group consisting of yttrium from group 3, titanium from group 4, vanadium from group 5, molybdenum from group 6, manganese from group 7, iron from group 8, cobalt from group 9, nickel from group 10, copper from group 11, zinc from group 12, aluminum from group 13, and tin from group 14.

5. The method of claim 4, wherein the concentration [Mi] of a metal Mi in the reaction mixture is between about 10 and about 750 milliequivalents (meq) per kg MM and the total concentration of metals Σ[Mi] is between about 20 and about 1500 meq per kg MM.

6. The method of claim 2, comprising isolating Mi from petroleum or heavy oil or resid.

7. The method of claim 2, wherein A1T1 is a protic solvent.

8. The method of claim 7, wherein A1T1 is water.

9. The method of claim 8, further comprising disposing the prepared feedstock in the form of a powder, granules, and/or pellets.

10. The method of claim 9, further comprising disposing the prepared feedstock in the form of a suspension, a slurry, a solution, or a melt.

11. The method of claim 9, further comprising selecting MM of a first kind (MM1), from the group consisting of nylons, polyesters, poly(ethyleneterephthalate), polyurethanes, polyurethane foams, lignin, lignocellulosic materials, renewable oils, biomass, and combinations thereof.

12. The method of claim 9, further comprising selecting MM of a second kind (MM2), from the group consisting synthetic MM2 (MM2/synth), tire rubber (MM2/tire), heavy components of petroleum oil (MM2/resid), and combinations thereof.

13. The method of claim 12, wherein MM2/synth comprises one or more materials whose formula is (CH₂CRR′)ₙ.

14. The method of claim 13, wherein R=H and R′=H, methyl, phenyl, and chloride corresponding to, respectively, polyethylene, polypropylene, polystyrene, and poly(vinylchloride).

15. The method of claim 13, wherein R=H or methyl and R′ is one or more taken from the group consisting of ethyl, vinyl, propyl, isopropyl, butyl, pentyl hexyl, cyclohexyl, phenyl, heptyl, and octyl.

16. The method of claim 12, wherein MM2/resid is the heavy fraction taken from petroleum by means of distillation or solvent deasphalting and includes one or more taken from the group consisting of asphaltenes, maltenes whose polarity and/or MW are elevated compared with other maltenes in the petroleum, and the vacuum residue generated in oil refineries by the vacuum distillation unit.

17. The method of claim 1, further comprising contacting the prepared feedstock in the flowpath or in the reactor with a first agent of a second type (A1T2) to obtain a premixture, wherein A1T2 is suitable to facilitate the disaggregation or dissolution of MM in the prepared feedstock and render the MM susceptible to undergoing Reactions1.

18. The method of claim 17, wherein A1T2 is a hydrocarbon.

19. The method of claim 18, wherein the hydrocarbon comprises one or more compounds selected from the group consisting of alkanes and cycloalkanes, which have the general formulas CₙH₂ₙ₊₂ and CₙH₂ₙ, respectively, and n is between about 5 and 20.

20. The method of claim 18, wherein the hydrocarbon comprises alkylbenzenes bearing one or more alkyl substituents, said substituents including one or more selected from the group consisting of methyl, ethyl, propyl, and butyl.

21. The method of claim 12, wherein the lower-MW fragments obtained by Reactions1 contain reactive functionality capable of undergoing undesirable reactions, which are quenched by the operation of hydrogen equivalents [H] in third desirable reactions (Reactions3).

22. The method of claim 21, further comprising generating [H] from a third agent (A3) added to the first reaction mixture or to Product1, wherein A3 comprises one or more materials with the with general formula CᵤHᵥO_{w} and undergoes fourth desirable reactions (Reactions4).

23. The method of claim 22, wherein Reactions4 comprises aqueous reforming in which A3 react with water to yield carbon dioxide and [H] according to the equation, CᵤHᵥO_{w}+(2u−w)H₂O→u CO₂+(4u+v−2w) [H].

24. The method of claim 22, wherein A3 comprises third agents of a first type (A3T1) including one or more materials with the general formulas (Cₓ(H₂O)_{y})ₙ, (CH₂O)ₙ, (C₆H₁₀O₅)ₙ, C₁₂H₂₂O₁₁, CₓH₂ₓ₊₂O_{y}, and (CᵤHᵥO_{w})ₙ, which include monosaccharides, cellulose, alcohols, diols, triols, tetraols, sorbitol, sorbitan, poly(vinyl alcohol), and lignin.

25. The method of claim 22, wherein A3 comprises third agents of a second type (A3T2) including one or more polyoxyalkylene materials with the general formula RO(CₓH₂ₓO)ₙR, where n>2, x=1, 2, 3, and 4, and R=C_{y}H_{y+1} with y=0, 1, 2, 3, or 4.

26. The method of claim 22, wherein A3 comprises third agents of a third type (A3T3) including one or more materials including polyesters, polyester resins, and polyurethanes produced through reactions with polyhydric alcohols including of one or more materials taken from the group consisting of compounds with the formula HO(CₓH₂ₓO)ₙH and those with the formulas CₓH_{2x+2-y}(OH)_{y}, (CₓH₂ₓ)(OH)₂, (CₓH₂ₓ₋₂)(OH)₂, and hydroxyl-terminated polyoxyalkylene adducts thereof.

27. The method of claim 12, wherein T(range)1 is in the range from T1/min to T1/max, which are about 325° C. and 370° C., respectively, and t1 is between about 2 minutes and 250 minutes.

28. The method of claim 1, further comprising configuring the flowpath upstream of the reactor to heat contents flowing therethrough to a temperature of up to T1/min before being conveyed into the reactor.

29. The method of claim 16, further comprising heating a heavy fraction from Product3 containing A2 to T(range)6 in a range from T6/min to T6/max, which are about 370° C. and 395° C., respectively, for a time t6 of between about 2 minutes and 150 minutes.

30. A system for deconstructing macromolecules (MM) into lower molecular weight (MW) fragments in high yield by promoting first desirable reactions (Reactions1) that result in chemolytic scission of bonds in the backbone, chain, matrix, or network that defines the MM and obtain a first product mixture (Product1), the system comprising:
(a) a flowpath configured to receive a prepared feedstock containing MM;
(b) a reactor disposed in fluid communication with the flowpath, wherein the prepared feedstock is conveyed in a downstream direction through the flowpath toward the reactor;
(c) the system configured to contact the prepared feedstock in the flowpath or in the reactor with a first agent of a first type (A1T1) suitable for promoting Reactions1;
(d) the system configured to contact the prepared feedstock in the flowpath or in the reactor with a second agent (A2) suitable for promoting Reactions1 to obtain a first reaction mixture;
(e) the reactor configured to heat the first reaction mixture to a temperature range T(range)1 in the range between T1/min and T1/max for a length of time t1 to obtain a product mixture Product1;
(f) the system configured to select A1T1 and T(range)1 in respect of MM chemistry and kind, which determines MM susceptibility to undergo Reactions1; and
(g) the system configured to select the total amount of A1T in the first reaction mixture and configuring the reactor to control the total pressure therein to establish amounts of A1T1 that exist in the liquid and gas phases when the first reaction mixture is heated in the reactor to T(range)1, where the amounts of A1T1 in the two phases are selected in respect of MM chemistry and kind and are appropriate to support Reactions1.
